(19) United States
(12) Patent Application Publication
(10) Pub. No.: US 2021/0069375 A1
(43) Pub. Date: Mar. 11, 2021

(54) Extracellular Matrices, Uses thereof, and Method for Making Extracellular Matrices

(71) Applicant: Gemstone Biotherapeutics LLC, Baltimore, MD (US)

(72) Inventors: Emily P. ENGLISH, Baltimore, MD (US); Matthew DAVENPORT, Baltimore, MD (US); Janty SHOGA, Baltimore, MD (US)

(21) Appl. No.: 16/958,553

(22) PCT Filed: Dec. 28, 2018
(86) PCT No.: PCT/US2018/067881

§ 371 Date: Jun. 26, 2020

Related U.S. Application Data
(60) Provisional application No. 62/611,381, filed on Dec. 28, 2017.
(60) Provisional application No. 62/682,011, filed on Jun. 7, 2018.

(51) Int. Cl. A61L 27/20; A61K 47/61; A61K 31/41; A61K 49/00; A61L 27/54; A61P 17/02; A61K 47/60; A61K 47/58

(52) U.S. Cl. CPC A61L 27/20; A61K 47/61; A61K 31/41; A61K 49/0054; A61L 2300/436; A61P 17/02; A61K 47/60; A61K 47/58; A61K 49/0043; A61L 27/54

(57) ABSTRACT

The invention is directed to extracellular matrix replacement (EMR)-drug conjugates or EMR-fluorescent label conjugates, to methods of making the EMR-drug conjugates or EMR-fluorescent label conjugates, to pharmaceutical compositions comprising the EMR-drug conjugates or EMR-fluorescent label conjugates, and to methods of treating wounds using the EMR-drug conjugates or EMR-fluorescent label conjugates. The invention is also directed to cure-in-place (CIP) EMRs, to methods of making the CIP EMRs, to pharmaceutical compositions comprising the CIP EMRs, and methods of treating wounds using the CIP EMRs.

This application claims the benefit under 35 U.S.C. 119(e) of provisional application 62/611,381 filed Dec. 28, 2017 and provisional application 62/682,011 filed Jun. 7, 2018, the disclosures of which are incorporated herein by reference in their entirety.

### BACKGROUND

In developed countries, aging populations and escalating rates of diabetes and obesity have led to the prevalence of chronic wounds. Chronic wounds are estimated to affect more than 6.5 million patients in the United States alone, and the annual healthcare burden associated with their treatment is estimated to be more than $25 billion. Chronic wounds have irregular boundaries and vary greatly in size, shape, and depth. Although the market is flooded with products for treating these chronic wounds, current products are expensive and show limited clinical effectiveness. Further, current products offer little capability to fit the precise shape and size of a wound bed.

Normal wound healing requires interactions of numerous cell types, soluble mediators, and the extracellular milieu to proceed through the wound healing cascade: inflammation, re-epithelialization, angiogenesis, tissue formation, wound contraction, and tissue remodeling. Chronic wounds remain stalled in the inflammatory stage.

One type of chronic wound is the diabetic ulcer, which is painful for diabetic patients and requires expensive long-term treatment. Diabetic ulcers often remain unhealed for over eight weeks and frequently reoccur. In the worst circumstances, diabetic ulcers lead to limb amputations. For example, diabetic foot ulcers precede 84% of all diabetes-related lower-leg amputations. In addition, these diabetic ulcers often recur, generally because they are unable to progress through the stages of normal wound healing. Diabetic patients suffer from macrovascular disease and microcirculatory deficiencies that reduce capillary size, impair endothelial function, and cause abnormal blood flow. These factors, combined with neuropathy, may render diabetic patients susceptible to pressure forces in their numbed feet, which may lead to foot ulcers and eventually to non-healing chronic wounds.

A significant and common problem associated with chronic wounds is infection, which contributes to their chronicity. Current treatment for non-healing chronic diabetic foot ulcers includes debridement followed by application of bioactive dressings and skin substitutes. These bioactive dressings and skin substitutes are expensive, require specialized training for application, have a short shelf life, and are unable to be further processed to incorporate active pharmaceutical ingredients. Because chronic wounds have been difficult to heal and have a high rate of recurrence, the effective treatment of chronic wounds remains an unmet challenge.

### SUMMARY OF THE INVENTION

The invention described herein is directed to extracellular matrix replacement (EMR)-comprising a pharmaceutically active compound, e.g., a small molecule/drug. In some embodiments the drug is conjugated to the EMR, (“EMR-drug conjugates”) and in some embodiments the drug is trapped within the spaces/pores of the EMR (“interstitial EMR drug compositions”). The invention described herein is also directed to methods of making the EMRs comprising the drugs, to pharmaceutical compositions comprising the EMRs comprising the drugs, and to methods of treating wounds using the EMRs comprising the drugs.

The invention described herein is also directed to fluorescently labeled EMRs, to methods of making the fluorescently labeled EMRs, to pharmaceutical compositions comprising the fluorescently labeled EMRs, and to methods of treating wounds using the fluorescently labeled EMRs.

The invention described herein is also directed to cure-in-place EMRs, to methods of making the cure-in-place EMRs, to pharmaceutical compositions comprising the cure-in-place EMRs, and methods of treating wounds using the cure-in-place EMRs. The cure-in-place EMRs of this invention effectively conform to the size, shape, and depth of a wound bed thereby filling the wound bed and further enhancing wound healing.

### BRIEF DESCRIPTION OF THE FIGURES

FIG. 1. Scheme for incorporating polyethylene(glycol)diacrylate into dextramate and for incorporating polyethylene(glycol)diacrylate and acrylate-polyethylene(glycol)-succinimidyl valeric acid into dextramate.

FIG. 2A. Scheme for reacting 5-fluoresceinamine with an EMR.

FIG. 2B. Scheme for reacting 5-fluoresceinamine with an EMR.

FIG. 3A. Scheme for reacting valsartan with polyethylene(glycol)acrylate.

FIG. 3B. Scheme for reacting a small molecule comprising a carboxylic acid group with polyethylene(glycol)acrylate.

FIG. 4A. Scheme for incorporating polyethylene(glycol)diacrylate and a valsartan-substituted polyethylene(glycol)acrylate into dextramate.

FIG. 4B. Scheme for incorporating polyethylene(glycol)diacrylate and a small molecule-substituted polyethylene(glycol)acrylate into dextramate.

FIG. 5A. Fluorescein-labeled gel imaged immediately after labeling.

FIG. 5B. Fluorescein-labeled gel imaged after labeling and after washing.

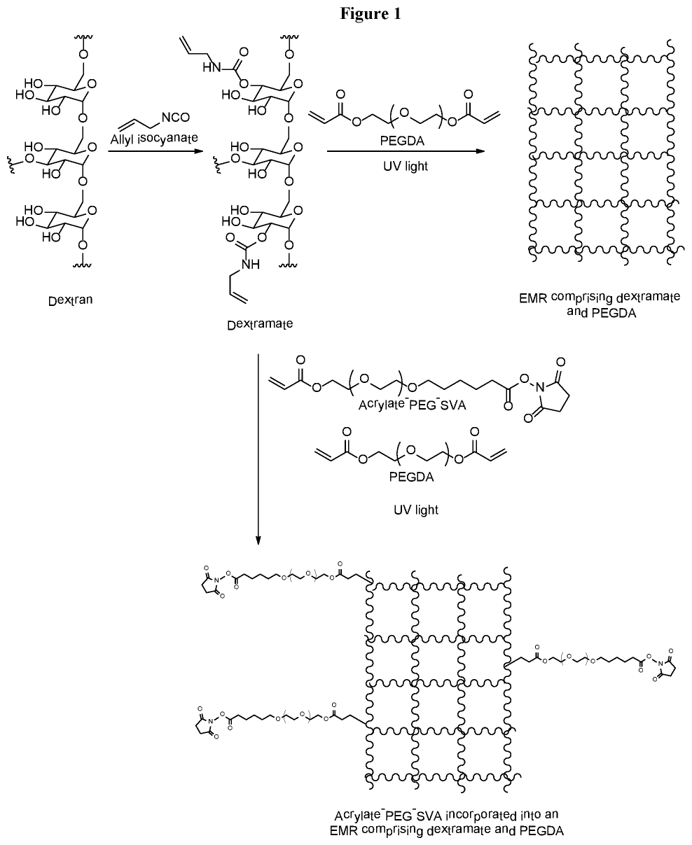

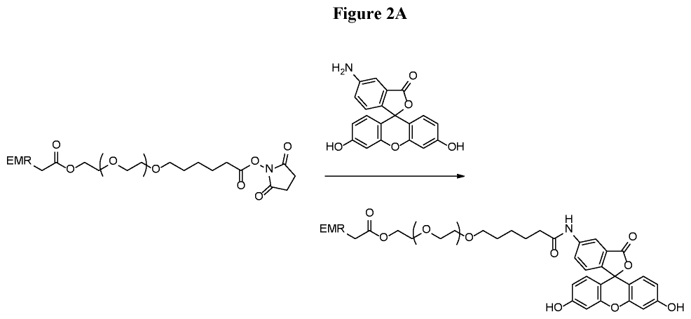

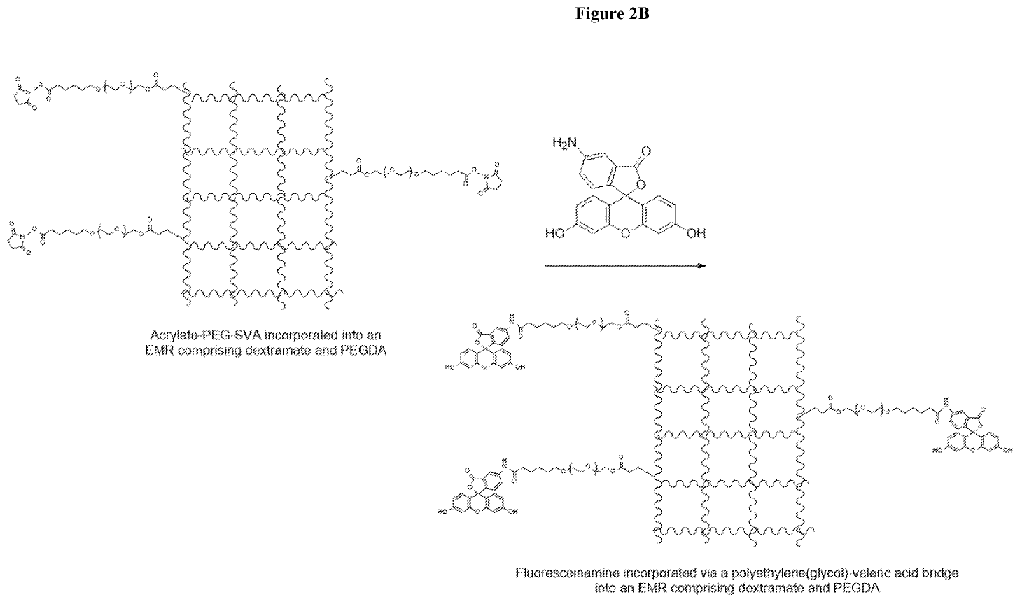

### DETAILED DESCRIPTION OF THE INVENTION

Described herein are EMRs, biocompatible hydrogels, that are useful in wound healing biomaterials because of their unique properties including physiochemical tunability, biocompatibility, degradability, and hydrophilicity. These properties allow the EMRs to provide a moist environment to the wound bed, promote autolysis, and adsorb wound exudate while functioning as a barrier to further trauma. The EMRs described herein provide a scaffold that stimulates cell infiltration, elicits an early and efficient inflammatory response, and recruits the necessary cell types for rapid revascularization and granulation tissue formation with complete scar-free skin regeneration and hair regrowth.

The EMRs described herein are made from at least one polymer. The EMRs are prepared by mixing one or more polymerizable entities (e.g., unfunctionalized glucans, e.g., unfunctionalized dextrans, and functionalized glucans, e.g. functionalized dextrans, and acrylate-comprising compounds) in aqueous solution. These polymerizable entities are then cured using UV light and/or visible light to generate the EMR. In some embodiments, once the EMR is generated, it is swelled in water, packaged, and sterilized. Clinicians can then apply the EMR as a patch by cutting it to size and placing it directly in the wound bed.

### EMRs Comprising Drugs

The EMRs of this invention enhance wound healing. In some embodiments the EMRs of this invention are combined with a pharmaceutically active compound, which compound enhances wound healing. Pharmaceutically active compounds that are suitable for use in EMRs of the invention described herein include compounds that are suitable for administration to a subject and provides a biological activity or other direct effect that enhances wound healing, e.g., small molecules/drugs, including an angiotensin receptor blocker (ARB), antibiotics, and analgesics.

The EMR and the drugs can be combined in different ways. In some embodiments the drug is trapped within the interstices of the EMR, producing an “interstitial” EMR-drug composition. In other embodiments, the drug is covalently bound to the EMR, producing an EMR-drug conjugate.

In an embodiment of this invention, the drug of the interstitial EMR-drug composition is any pharmaceutically active compound that promotes wound healing, prevents wound infection, and/or provides pain relief. Examples of suitable pharmaceutically active compounds for use in the interstitial EMR-drug compositions are small molecules, e.g., angiotensin receptor blockers (ARBs), antibiotics, and analgesics. Examples of suitable ARBs are valsartan, olmesartan, azilsartan, eprosartan, candesartan, telmisartan, carboxylosartan, losartan, and irbesartan. Suitable examples of antibiotics are penicillins, cephalosporins, sulfonamides, tetracyclines, aminoglycosides, glycopeptides, and macrolides. Examples of suitable analgesics are non-steroidal inflammatory drugs (e.g., aspirin, salicylic acid, ketorolac, diclofenac, indomethacin, ibuprofen, ketoprofen, and naproxen), opioids, opiates, gabapentin, and pre-gabalin.

In an embodiment of this invention, the drug of the EMR-drug conjugate is any pharmaceutically active compound that comprises a carboxylic acid group. Examples of such pharmaceutically active compounds include small molecules, e.g. angiotensin receptor blockers (ARBs), antibiotics, and analgesics, that comprise a carboxylic acid group. Examples of ARBs comprising a carboxylic acid group are valsartan, olmesartan, azilsartan, eprosartan, candesartan, telmisartan, carboxylosartan, and irbesartan. Examples of suitable antibiotics comprising a carboxylic acid group are penicillins and cephalosporins. Examples of analgesics comprising a carboxylic acid group are gabapentin, pre-gabalin, aspirin, salicylic acid, ketorolac, diclofenac, indomethacin, ibuprofen, ketoprofen, and naproxen.

In an embodiment of this invention, the drug of the EMR-drug conjugate is any pharmaceutically active compound that comprises a functional group (e.g., an alcohol group, an ester group) that can be transformed into a carboxylic acid group. Examples of pharmaceutically active compounds that comprise a functional group that can be transformed into a carboxylic acid group are ARBs (e.g., losartan).

In an embodiment of this invention, the drug of the interstitial EMR-drug composition or the EMR-drug conjugate is an ARB, e.g., valsartan, olmesartan, azilsartan, eprosartan, candesartan, telmisartan, carboxylosartan, irbesartan, losartan, or mixtures thereof. In an embodiment of this invention, the interstitial EMR-drug composition or the EMR-drug conjugate comprising an ARB accelerates wound closure.

In an embodiment, the interstitial EMR-drug composition or EMR-drug conjugate of the invention closely match the physical properties of unfunctionalized EMRs, including the swelling properties, the stiffness, the porosity, and the oxygen permeability. Suitable examples of unfunctionalized EMRs, as well as methods for preparing and characterizing unfunctionalized EMRs may be found in, e.g., U.S. Pat. Nos. 8,900,868 and 9,655,844, U.S. Pre-Grant Publication Nos. 2013/0102531 and 2015/0174154, and Sun et al., PNAS 2011, 108, 20976-20981 and Shen et al., Acellular Hydrogels for Regenerative Burn Wound Healing: Translation from a Porcine Model, Journal of Investigative Dermatology 2015. The properties of the EMR-drug conjugates, like the properties of unfunctionalized EMRs, are determined through conventional methods. See, e.g., Sun et al., PNAS 2011, 108, 20976-20981, which is incorporated in its entirety by reference.

For example, the swelling ratios of the interstitial EMR-drug compositions or EMR-drug conjugates are determined via gravimetric analysis to evaluate the capacity of the interstitial EMR-drug compositions or EMR-drug conjugates to absorb water (as a surrogate for wound exudate) and desorb water (as a surrogate for wound hydration). A moist environment promotes autolytic debridement.

As another example, critical stiffness of the EMR is required to maintain the integrity of the wound bed for the healing of self-contracting wounds without compromising the properties of the EMR. Standard mechanical measurements of the elastic modulus are collected for the interstitial EMR-drug compositions or EMR-drug conjugates using a rheometer. A suitable Young's modulus for the interstitial EMR-drug compositions or EMR-drug conjugates of the invention is between about 10 Pa and about 2000 Pa, between about 10 Pa and about 1500 Pa, between about 10 Pa and about 1000 Pa, between about 10 Pa and about 500 Pa, and between about 10 Pa and about 250 Pa.

As another example, the porosity and morphology of the EMRs, which are directly related to the crosslinking density, affects cell infiltration, tissue ingrowth and degradation rate of the polymers of the EMR. The morphology and porosity of all interstitial EMR-drug compositions or EMR-drug conjugates can be determined using scanning electron microscopy to provide a representative view of the microstructure of the interstitial EMR-drug compositions or EMR-drug conjugates. A suitable oxygen permeability for the interstitial EMR-drug compositions or EMR-drug conjugates is full oxygen permeability under normoxic conditions.

As another example, oxygen transfer facilitates wound healing because it increases granulation tissue formation, epithelialization, and fibroblast recruitment and promotes wound contraction. The interstitial EMR-drug compositions or EMR-drug conjugates must be oxygen permeable to enhance wound healing and prevent anaerobic bacterial infections, which is thought to occur at the interface of the wound bed and oxygen impermeable wound dressings. The rate of oxygen transfer through hydrated interstitial EMR-drug compositions or EMR-drug conjugates of various thicknesses is measured continuously using oxygen sensors under both physiological and hypoxic conditions. Monitoring oxygen permeability over time provides a temporal profile of oxygen transfer through the EMR.

In all of these characterization steps, the interstitial EMR-drug compositions or EMR-drug conjugates of the invention are compared to unfunctionalized EMR as a control. Unfunctionalized EMRs are well-characterized and produced by known methods. See, e.g., U.S. Pat. Nos. 8,900,868 and 9,655,844, incorporated herein in their entirety. See also U.S. Pre-Grant Publication Nos. 2013/0102531 and 2015/0174154, incorporated herein in their entirety. See also Sun et al., PNAS 2011, 108, 20976-20981 and Shen et al., Acellular Hydrogels for Regenerative Burn Wound Healing: Translation from a Porcine Model, Journal of Investigative Dermatology 2015, incorporated herein in their entirety. The properties of the unfunctionalized EMRs include an elastic modulus of approximately 1.5 kPa, pore sizes of ˜10 μM, and full oxygen permeability under normoxic conditions.

The interstitial EMR-drug composition or the EMR-drug conjugate is applied to a chronic wound (e.g., a diabetic ulcer), which: (1) provides mechanical support by mimicking the mechanical properties of tissue; (2) increases the rate of wound healing and closure by increasing cell migration and revascularization of the wound site; and (3) enhances the rate of wound healing and closure, as compared to the EMR without the drug, by releasing the drug into the wound for a prolonged period.

Wound healing involves the mediation of many initiators that are critical for tissue repair. For example, fibroblasts are recruited to the wound area, where they proliferate and aid in remodeling the extracellular environment and wound contraction.

A scratch wound assay may be used to evaluate basic fibroblast cell recruitment induced by interstitial EMR-drug compositions or EMR-drug conjugates. Briefly, fibroblast cells are grown to confluence and a thin wound is created by scratching the monolayer with a pipette tip. The interstitial EMR-drug composition or EMR-drug conjugate is then applied to the thin wound at various time points to assess its effect on fibroblast cell recruitment. For example, the EMR-drug conjugate or interstitial EMR-drug composition may be applied immediately after scratching the fibroblasts, or application of the EMR-drug conjugate or interstitial EMR-drug composition may be delayed for minutes or hours thereafter. The migratory speed and number of fibroblasts infiltrating the wound area in the presence of varying amounts of the EMR-drug conjugate or interstitial EMR-drug compositions are counted using time lapse light microscopy and compared to controls containing no EMR or an unfunctionalized EMR. Together, these data provide insight into the ability of the EMR-drug conjugate or interstitial EMR-drug compositions to promote fibroblast recruitment. Furthermore, any early indicators of potential cytotoxicity of the EMR-drug conjugate or interstitial EMR-drug compositions are discovered in these assays. This assay can also be performed with mammalian endothelial cells (e.g., human endothelial cells), which are essential for the neovascularization of the wound bed to provide critical nutrients and oxygen to the damaged tissue.

Cytotoxicity of the EMR-drug conjugates or interstitial EMR-drug compositions may be evaluated by culturing human fibroblasts with the EMR-drug conjugates or interstitial EMR-drug compositions of the invention in wells and then quantifying fibroblast viability, morphology, and proliferation in the presence of the EMR-drug conjugates or interstitial EMR-drug compositions of the invention. Cytotoxicity of the EMR-drug conjugates or interstitial EMR-drug compositions may also be evaluated by culturing other cell types (e.g., keratinocytes) with the EMR-drug conjugates and interstitial EMR-drug compositions of this invention in wells and then quantifying the viability, morphology, and proliferation of these other cell types in the presence of EMR-drug conjugates and interstitial EMR-drug compositions of this invention.

Cell morphology, fibroblast viability, and proliferation may be analyzed by any well-known method in the art, e.g., cell morphology may be analyzed via light microscopy, to identify any structural changes to the cell, and the fibroblast viability may be confirmed via a two-color fluorescence cell viability assay that visualizes both viable and non-viable cells using fluorescence microscopy or flow cytometry.

Fibroblast proliferation may be measured using a WST assay (commercially available WST-1 Cell Proliferation Assay kits are available from Cayman Chemical).

The EMR-drug conjugates and interstitial EMR-drug compositions of the invention stay intact for long enough to provide sufficient mechanical support for cells to migrate into the wound bed but degrade sufficiently to allow sustained drug release, further cell invasion, and tissue regeneration. In vitro degradation of the EMR in the EMR-drug conjugates and interstitial EMR-drug compositions is quantified using a neutrophil-like cell suspension using HL-60s differentiated in DMSO for five days is used. Samples of the inventive interstitial EMR-drug compositions or EMR-drug conjugates are incubated in these neutrophil-like cultures for about 12, 24, 36, 48, 60, and 72 hours, or anytime there between, after which the washed EMR-drug conjugates or interstitial EMR-drug compositions are lyophilized, and the initial dry weight of the EMR-drug conjugates or interstitial EMR-drug compositions are compared to the post-degraded dry weight of the EMR-drug conjugates or interstitial EMR-drug compositions. This provides the culture-based degradation kinetics for each formulation of interstitial EMR-drug composition or EMR-drug conjugates. Drug release is also evaluated in this experiment, and the amount of drug released into the culture media is quantified by HPLC/MS (high performance liquid chromatography/mass spectrometry) or ELISA. Methods for quantifying drugs via HPLC/MS are well known in the art.

For assaying and comparing the release kinetics of the drug from EMR-drug conjugates or interstitial EMR-drug compositions in cell culture, a panel of endogenous enzymes that are expected to release the drug from the EMR scaffold is selected. This panel of enzymes includes, e.g., proteinases, proteases, esterases, and glycosidases such as those that have known roles in extracellular matrix remodeling during wound healing (e.g., matrix metalloproteinase, gelatinases and collagenases). The EMR-drug conjugates or interstitial EMR-drug compositions are incubated with a single enzyme or with a mixture of enzymes selected from the panel of enzymes. Drug release is measured over time on incubation of the EMR-drug conjugates or interstitial EMR-drug compositions with the single enzymes or mixture of enzymes.

Without wishing to be bound by theory, it is contemplated that the drug component of the EMR-drug conjugates or interstitial EMR-drug compositions is released as the polymers of the EMR component of the EMR-drug conjugates or interstitial EMR-drug compositions are digested by endogenous enzymes at the wound site. The effectiveness of treating a wound with the EMR-drug conjugates or interstitial EMR-drug compositions may be measured using methods known in the art and may be compared to a control, e.g., treating the wound with the drug alone (e.g., an ARB not combined with an EMR) and an EMR without the drug.

The EMR-drug conjugates and interstitial EMR-drug compositions of the invention ensure patient compliance. Adherence to a regimen of cleaning and applying a dressing or drug to a wound, as is required in conventional would treatment, is difficult for diabetic patients in poor health. The EMR-drug conjugates and inventive interstitial EMR-drug compositions of the invention provide mechanical support to the wound, increase cell migration and revascularization of the wound site, and provide drug release into the wound for a prolonged time period. The prolonged time period is at least 5 days, preferably at least 7 days, preferably at least 8 days, preferably at least 10 days, preferably at least 12 days, preferably at least 14 days, preferably at least 20 days. Thus, with the EMR-drug conjugates and inventive interstitial EMR-drug compositions of the invention, patients do not have to clean and apply a dressing or drug to a wound daily, which leads to increased patient compliance and faster healing.

This invention provides a method for preparing the interstitial EMR-drug composition of the invention, comprising: (a) mixing a pharmaceutically active compound and a polymerizable entity; and (b) curing the mixture of step (a) with UV light and/or visible light. Examples of suitable polymerizable entities include unfunctionalized glucans (e.g., unfunctionalized dextrans) and functionalized glucans (e.g., unfunctionalized dextrans) and functionalized dextrans as described herein.

In one embodiment of the method for preparing the interstitial EMR-drug composition of the invention, the pharmaceutically active compound and the polymerizable entity are mixed together with a cross-linking catalyst selected from a UV-crosslinking catalyst or a visible light-crosslinking catalyst. Examples of suitable UV-crosslinking catalysts are Irgacure catalysts (e.g., Irgacure 2959) and lithium phenyl-2,4,6-trimethylbenzoylphosphinate (LAP). An example of a suitable visible-light cross-linking catalysts is eosin-Y.

In one embodiment of the method for preparing the interstitial EMR-drug composition of the invention, the pharmaceutically active compound, the polymerizable entity, and the cross-linking catalyst are further mixed together with an acrylate-comprising compound (e.g., polyethylene(glycol)diacrylate, polyethylene(glycol)acrylate, and acrylate-polyethylene(glycol)-succinimidyl valeric acid).

This invention provides a method for preparing the EMR-drug conjugate of the invention, comprising: (a) conjugating a pharmaceutically active compound with a polymerizable entity; and (b) curing the product of step (a) with UV light and/or visible light. Examples of suitable polymerizable entities include unfunctionalized glucans (e.g., unfunctionalized dextrans) and functionalized glucans (e.g., functionalized dextrans) as described herein.

In one embodiment of the method for preparing the EMR-drug conjugate of the invention, the pharmaceutically active compound and the polymerizable entity are mixed together with a cross-linking catalyst selected from a UV-crosslinking catalyst or a visible light-crosslinking catalyst. Examples of suitable UV-crosslinking catalysts are Irgacure catalysts (e.g., Irgacure 2959) and lithium phenyl-2,4,6-trimethylbenzoylphosphinate (LAP). An example of a suitable visible-light cross-linking catalysts is eosin-Y.

In one embodiment of the method for preparing the EMR-drug conjugate of the invention, the pharmaceutically active compound, the polymerizable entity, and the cross-linking catalyst are further mixed together with an acrylate-comprising compound (e.g., polyethylene(glycol)diacrylate, polyethylene(glycol)acrylate, and acrylate-polyethylene(glycol)-succinimidyl valeric acid).

In one embodiment of the method for preparing the interstitial EMR-drug composition or the EMR-drug conjugate of the invention, the polymerizable entity is unfunctionalized glucan. Suitable unfunctionalized glucans that may be used in the interstitial EMR-drug compositions or the EMR-drug conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the interstitial EMR-drug composition or the EMR-drug conjugate of the invention, the polymerizable entity is unfunctionalized dextran, which has the following structure:

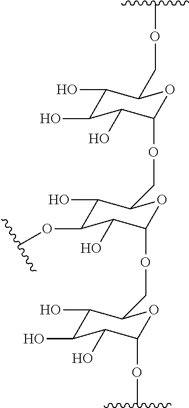 [SMILES: *OCC1OC(OCC2OC(OCC3OC(O*)C(O)C(O)C3O)C(O)C(O*)C2O)C(O)C(O)C1O]

Suitable unfunctionalized dextrans that may be used in the interstitial EMR-drug compositions or the EMR-drug conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the interstitial EMR-drug composition or the EMR-drug conjugates of the invention, the polymerizable entity is a functionalized glucan. Suitable functionalized glucans are glucans that are functionalized with polymerizable compounds, such as ethylamine, allyl carbamate, and mixtures thereof. Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the interstitial EMR-drug compositions and EMR-drug conjugates of the invention, suitable functionalized glucans have a degree of substitution between about 0.01 and about 0.9, between about 0.02 and about 0.8, between about 0.05 and about 0.7, between about 0.1 and about 0.6, between about 0.15 and about 0.5.

In one embodiment of the method for preparing the interstitial EMR-drug composition or the EMR-drug conjugates of the invention, the polymerizable entity is a functionalized dextran. Suitable functionalized dextrans are dextrans that are functionalized with polymerizable compounds, such as ethylamine, allyl carbamate, and mixtures thereof. Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the interstitial EMR-drug compositions and EMR-drug conjugates of the invention, suitable functionalized dextrans have a degree of substitution between about 0.01 and about 0.9, between about 0.02 and about 0.8, between about 0.05 and about 0.7, between about 0.1 and about 0.6, between about 0.15 and about 0.5.

In a preferred embodiment of the interstitial EMR-drug compositions and EMR-drug conjugates of the invention, the polymerizable entity is dextramate. Dextramate is a dextran that has been reacted with allyl isocyanate molecules to produce a dextran that is functionalized with allyl carbamate groups. In some embodiments of the invention, the degree of substitution in the dextramate is between about 0.01 and about 0.9, between about 0.02 and about 0.8, between about 0.05 and about 0.7, between about 0.1 and about 0.6, or between about 0.15 and about 0.5. The degree of substitution refers to the degree of hydroxyl groups on dextran that have reacted with allyl isocyanate molecules (i.e., the degree of hydroxyl groups on dextran that are substituted with allyl carbamate groups).

For use in the interstitial EMR-drug compositions and EMR-drug conjugates of the invention, suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da.

One embodiment of the invention is an interstitial EMR-drug composition or an EMR-drug conjugate wherein the EMR component comprises a mixture of a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One preferred embodiment of the invention is an interstitial EMR-drug composition or an EMR-drug conjugate wherein the EMR component comprises a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One embodiment of the invention is an interstitial EMR-drug composition or an EMR-drug conjugate wherein the drug component makes up about 0.001% to about 25% of the total weight of the interstitial EMR-drug composition or an EMR-drug conjugate, about 0.01% to about 10% of the total weight of the interstitial EMR-drug composition or an EMR-drug conjugate, or about 0.1% to about 5% of the total weight of the interstitial EMR-drug composition or an EMR-drug conjugate.

In one embodiment, the EMR-drug conjugate has the following formula (I), wherein a pharmaceutically active compound is covalently linked to the EMR component:

Q-X—Y (I),

Q is the pharmaceutically active compound, X is a linker group, and Y is an EMR.

One embodiment of the invention is an EMR-drug conjugate of formula (I) wherein the pharmaceutically active compound is a small molecule comprising a carboxylic acid group.

This invention provides a method for preparing the EMR-drug conjugate of the invention, comprising:

(a) reacting a small molecule of formula (XVIII) with a compound of formula (XIXa) or formula (XIXb) to yield a compound of formula (XXa) or (XXb):

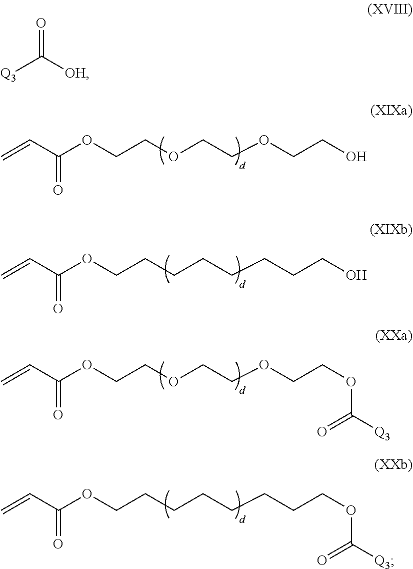

wherein Q₃ is a residue of any small molecule (e.g., a small molecule selected from the group consisting of ARBs, antibiotics, and analgesics) and wherein d is 0 or an integer between 1 and 500, between 1 and 250, between 1 and 200, between 1 and 100, between 1 and 50, or between 1 and 10;

(b) reacting the compound of formula XXa or formula XXb with acryl carbamate groups on a dextran of formula X:

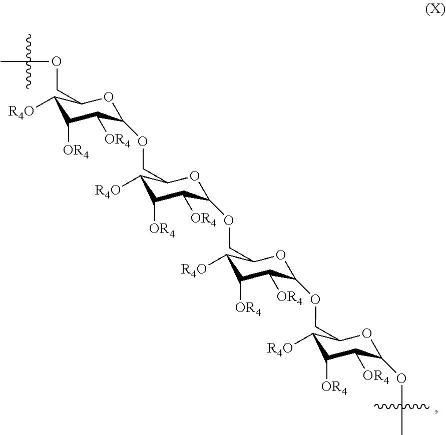

wherein R₄ is H, allyl carbamate, or mixtures thereof;

(c) optionally adding to the product of step (b) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(d) curing the product of step (b) or step (c) with UV light and/or visible light.

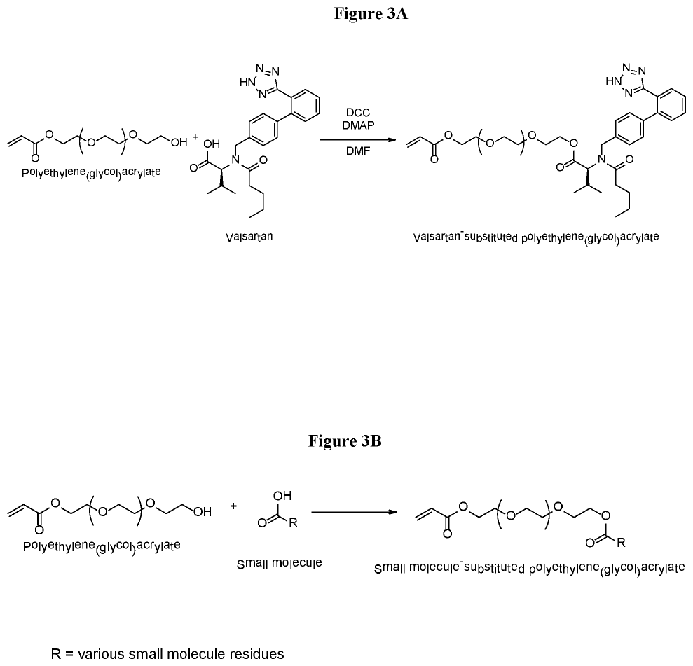

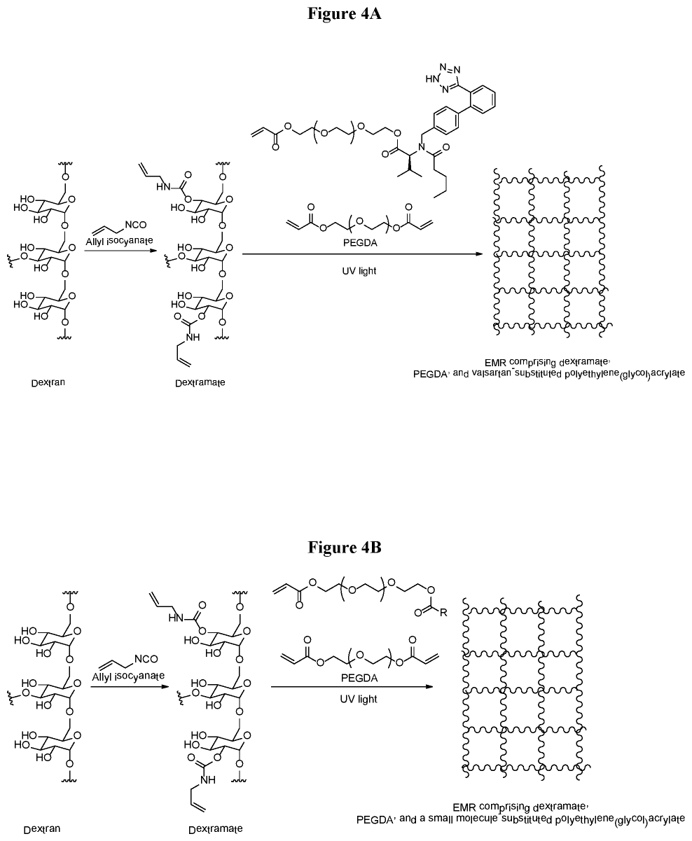

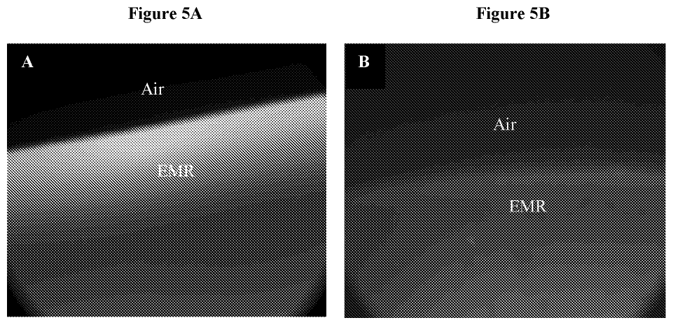

Step (b) of this method is illustrated in FIG. 3B. Steps (c) and (d) of this method are illustrated in FIG. 4B.

In one preferred embodiment of the method, Q₃ in formula (XVIII) is selected from the group consisting of

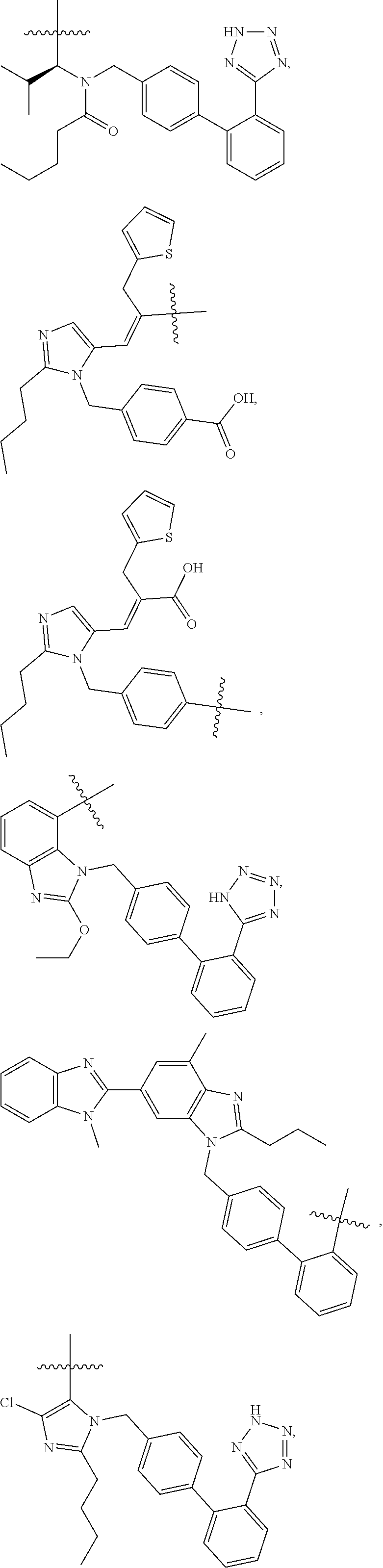

and mixtures thereof.

One embodiment of the invention is an EMR-drug conjugate of formula (I) wherein the pharmaceutically active compound is an ARB is selected from the group consisting of valsartan, olmesartan, azilsartan, eprosartan, candesartan, telmisartan, carboxylosartan, irbesartan, losartan, and mixtures thereof.

Another embodiment of the invention is an EMR-drug conjugate of formula (I) wherein

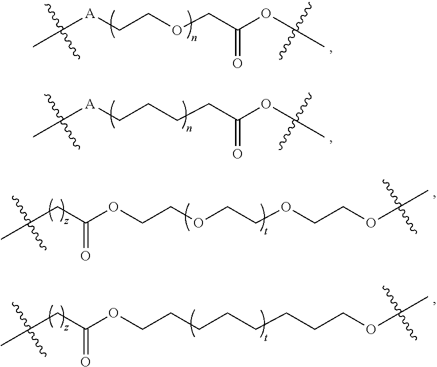

the linker group is selected from the group consisting of: a bond, and mixtures thereof, wherein A is O, S, or NH, n is 0 or an integer between 1 and 500, between 1 and 250, between 1 and 200, between 1 and 100, between 1 and 50, or between 1 and 10; t is 0 or an integer between 1 and 500, between 1 and 250, between 1 and 200, between 1 and 100, between 1 and 50, or between 1 and 10; and z is 0 or an integer between 1 and 10.

In one embodiment of the EMR-drug conjugate of formula (I), the pharmaceutically active compound is valsartan.

In some embodiments of the invention, the EMR-drug conjugate further comprises a pharmaceutically active compound that is trapped within the interstices of the EMR-drug conjugate. Examples of suitable pharmaceutically active compounds that may be trapped within the interstices of the EMR-drug conjugate ARBs, antibiotics, and analgesics.

This invention provides a method for preparing the EMR-drug conjugate of the invention, comprising:

(a) transforming an ARB with a structure of formula (IIa) into a compound of formula (IIb):

Q₁-OH (IIa)

Q₁-L (IIb),

wherein Q₁ is selected from the group consisting of:

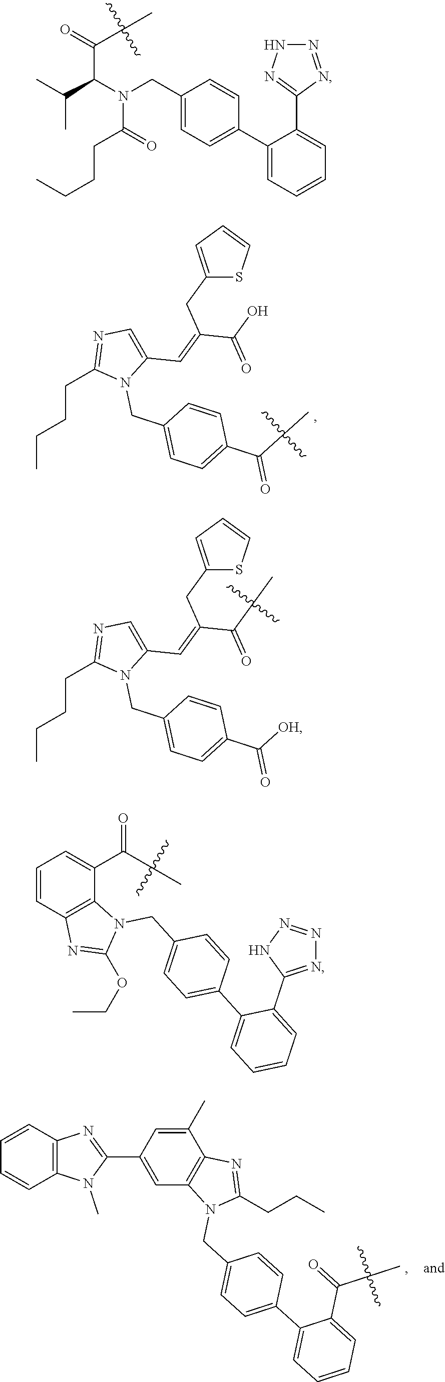

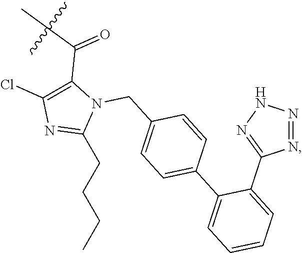

wherein L is selected from the group consisting of: —Cl, —Br, —I, and —OR₁, wherein R₁ is C₁-C₁₀ alkyl or

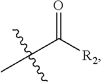

and wherein R₂ is C₁-C₁₀ alkyl;

(b) reacting the compound of formula (IIb) with a compound of formula (IIIa) or formula (IIIb) to yield a compound of formula (IVa) or (IVb):

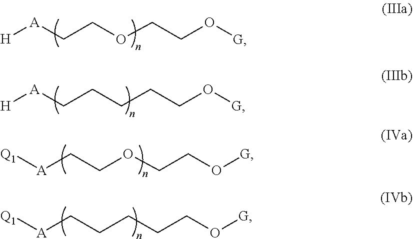

wherein G is a protecting group, A is O, S, or NH, n is 0 or an integer between 1 and 500, between 1 and 250, between 1 and 200, between 1 and 100, between 1 and 50, or between 1 and 10; and Q₁ is the same as defined in step (a);

(c) deprotecting the compound of formula (IVa) or formula (IVb) to yield a compound of formula (Va) or formula (Vb):

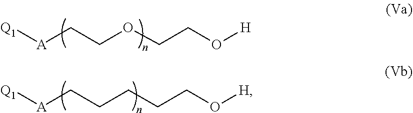

wherein Q₁ is the same as defined in step (a) and A and n are the same as defined in step (b);

(d) oxidizing the compound of formula (IVa) or formula (IVb) to yield a product of formula (VIa) or formula (VIb):

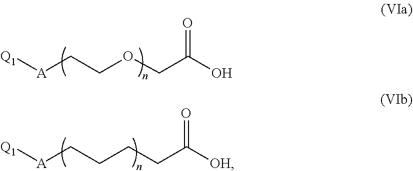

wherein Q₁ is the same as defined in step (a) and A and n are the same as defined in step (b);

(e) transforming the compound of formula (VIa) or formula (VIb) to yield a compound of formula (VIIa) or formula (VIIb):

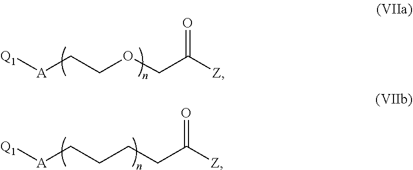

wherein Z is —Cl, —Br, —I, and —OR₃, wherein R₃ is C₁-C₁₀ alkyl, and wherein Q₁ is the same as defined in step (a) and A and n are the same as defined in step (b);

(f) reacting the compound of formula (VIIa) or formula (VIIb) with free hydroxyl groups on a polymerizable entity selected from the group consisting of unfunctionalized dextran, functionalized dextran, and mixtures thereof;

(g) optionally adding to the product of step (f) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(h) curing the product of step (f) or step (g) with UV light and/or visible light.

Another embodiment of the invention is a method for preparing the EMR-drug conjugate of the invention, comprising:

(a) oxidizing an ARB with a structure of formula (IIc)

Q₂-CH₂OH (IIc),

wherein Q₂ is

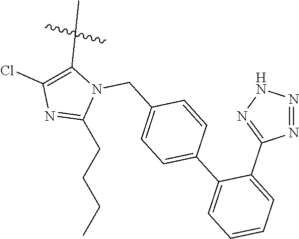 [SMILES: *c1c(Cl)nc(CCCC)n1Cc1ccc(-c2ccccc2-c2nn[nH]n2)cc1]

into a compound of formula (IIa-1):

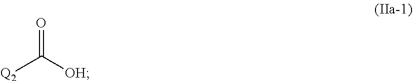

(b) transforming the compound formula (IIa-1) into a compound of formula (IIb-1):

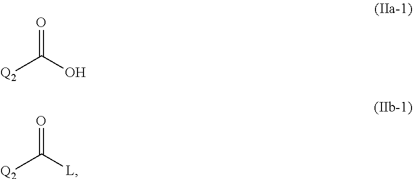

wherein Q₂ is the same as defined in step (a), wherein L is selected from the group consisting of —Cl, —Br, —I, and —OR₁, wherein R₁ is C₁-C₁₀ alkyl or

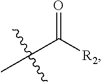

and wherein R₂ is C₁-C₁₀ alkyl;

(c) reacting the compound of formula (IIb-1) with a compound of formula (IIIa) or formula (IIIb) to yield a compound of formula (IVa-1) or (IVb-1):

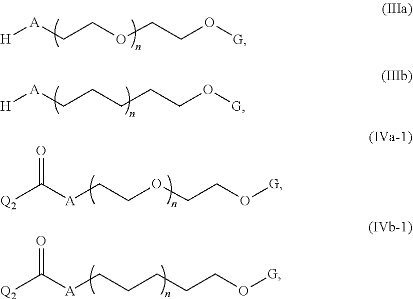

wherein G is a protecting group, A is O, S, or NH, n is 0 or an integer between 1 and 500, between 1 and 250, between 1 and 200, between 1 and 100, between 1 and 50, or between 1 and 10; and Q₂ is the same as defined in step (a)

(d) deprotecting the compound of formula (IVa-1) or formula (IVb-1) to yield a compound of formula (Va-1) or formula (Vb-1):

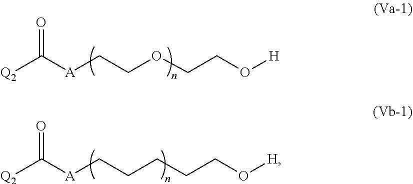

wherein Q₂ is the same as defined in step (a) and A and n are the same as defined in step (c);

(e) oxidizing the compound of formula (IVa-1) or formula (IVb-1) to yield a product of formula (VIa-1) or formula (VIb-1):

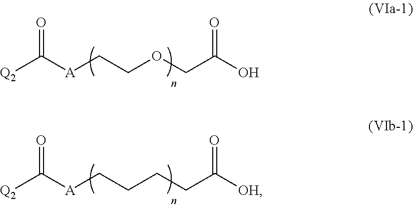

wherein Q₂ is the same as defined in step (a) and A and n are the same as defined in step (c);

(f) transforming the compound of formula (VIa-1) or formula (VIb-1) to yield a compound of formula (VIIa-1) or formula (VIIb-1):

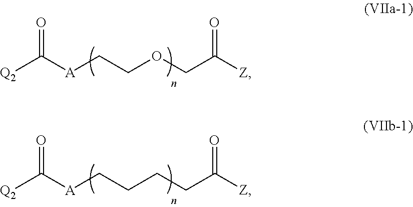

wherein Z is —Cl, —Br, —I, and —OR₃, wherein R₃ is C₁-C₁₀ alkyl, and wherein Q₂ is the same as defined in step (a) and A and n are the same as defined in step (c);

(g) reacting the compound of formula (VIIa-1) or formula (VIIb-1) with free hydroxyl groups on a polymerizable entity selected from the group consisting of unfunctionalized dextran, functionalized dextran, and mixtures thereof.

(h) optionally adding to the product of step (g) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(i) curing the product of step (g) or step (h) with UV light and/or visible light.

Another embodiment of the invention is a method for preparing the EMR-drug conjugate of the invention, wherein the protecting group G is selected from the group consisting of C₁-C₁₀ unbranched or branched alkyl; —SiMe₃; —SiEt₃; —Si(iPr)₃; —SiPh₃; —SiMe₂iPr; —SiMe₂Et; —SiEt₂iPr; and —CH₂-Ph, wherein the Ph is unsubstituted or substituted with at least one substituent selected from the group consisting of —OMe, —NO₂, —F, —Cl, —Br, —I, —CF₃, —SiMe₃, and —CN.

In an embodiment of the invention the EMR-drug conjugate comprises a functionalized dextran of formula (X):

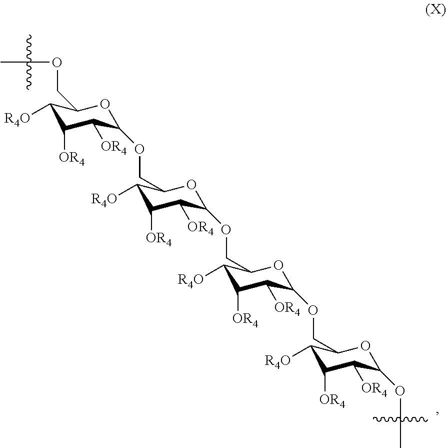

wherein R₄ is H, allyl carbamate, or mixtures thereof.

Another embodiment of the invention is a method for preparing the EMR-drug conjugate of the invention, comprising:

(a) reacting a compound of formula (IIa):

Q₁-OH (IIa),

wherein Q₁ is selected from the group consisting of:

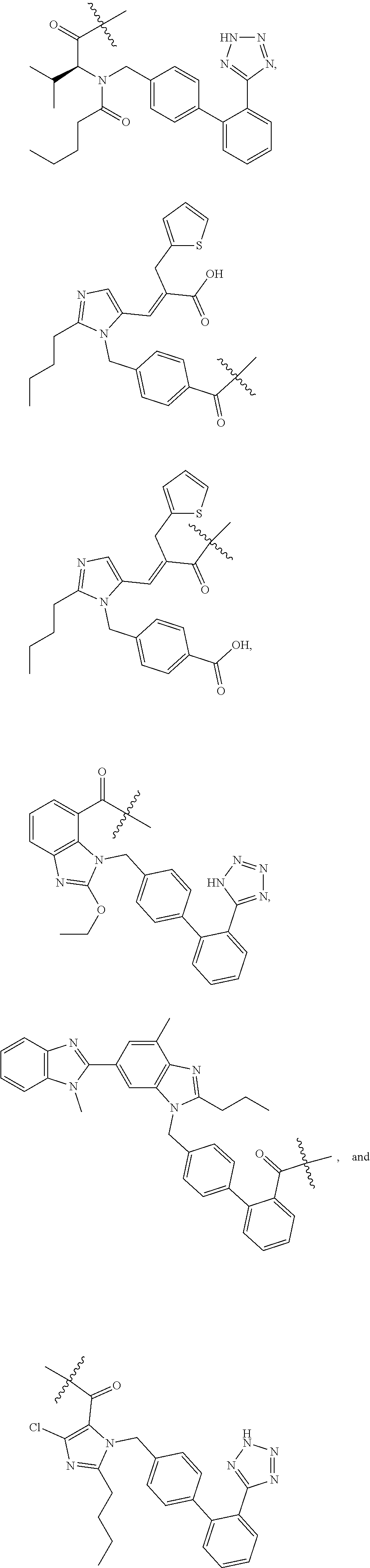

with a structure of formula (XIVa) or formula (XIVb):

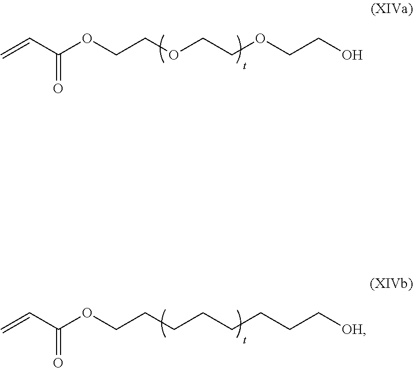

wherein t is 0 or an integer between 1 and 500, between 1 and 250, between 1 and 200, between 1 and 100, between 1 and 50, or between 1 and 10,

to yield a compound of formula (XVa) or (XVb):

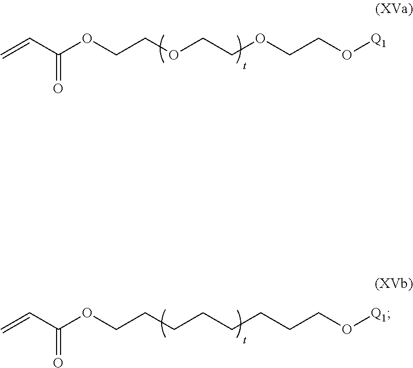

(b) mixing the compound of formula (XVa) or formula (XVb) with a compound of formula (X):

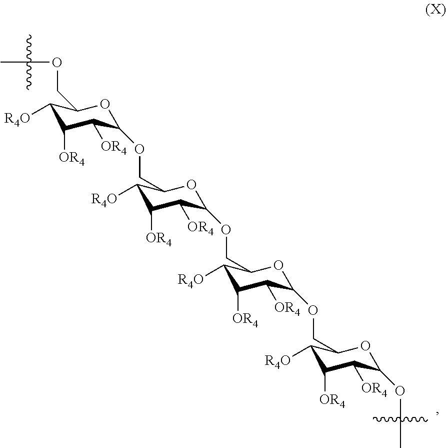

wherein R₄ is H, allyl carbamate, or mixtures thereof;

(c) optionally adding to the product of step (b) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(d) curing the product of step (b) or step (c) with UV light and/or visible light.

Another embodiment of the invention is a method for preparing the EMR-drug conjugate of the invention, comprising:

(a) transforming a compound of formula (XIVa) or formula (XIVb):

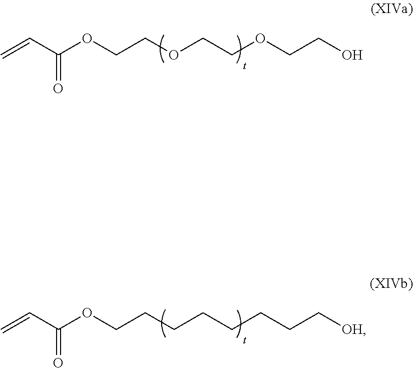

into a compound of formula (XIVa-1) or formula (XIVb-1):

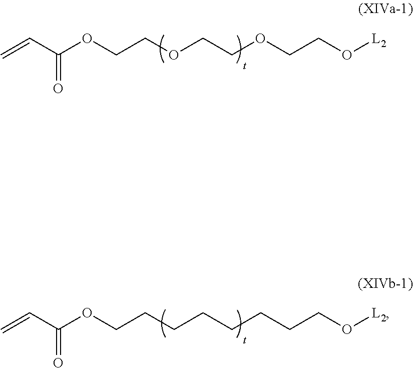

wherein t is 0 or an integer between 1 and 500, between 1 and 250, between 1 and 200, between 1 and 100, between 1 and 50, or between 1 and 10; and L₂ is a group selected from the group consisting of C₁-C₁₀ alkyl or

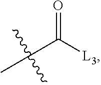

and wherein L₃ is C₁-C₁₀ alkyl;

(b) reacting the compound of formula (XIVa-1) or formula (XIVb-1) with a compound of formula (IIc):

Q₂-CH₂OH (IIc)

to yield a compound of formula (XIVa-2) or formula (XIVb-2):

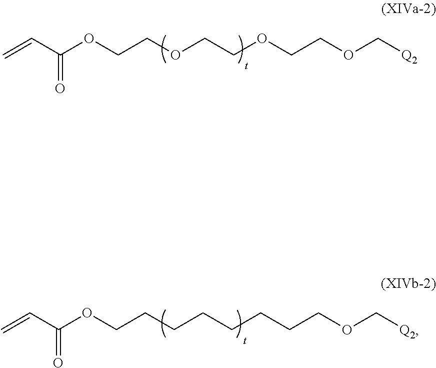

wherein Q₂ is

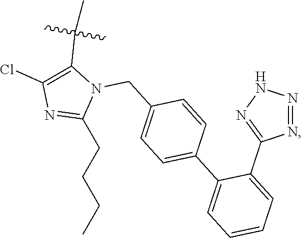 [SMILES: *c1c(Cl)nc(CCCC)n1Cc1ccc(-c2ccccc2-c2nn[nH]n2)cc1]

and wherein t and L₂ are as defined in step (a);

(c) mixing the compound of formula (XIVa-2) or formula (XIVb-2) with a compound of formula (X):

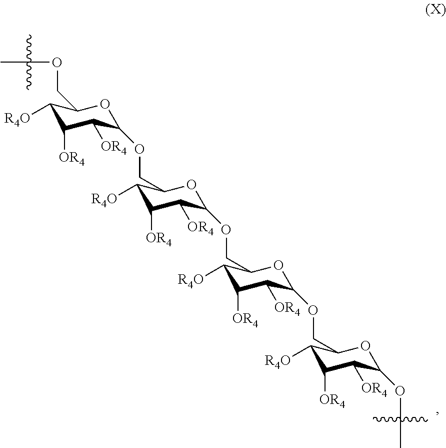

wherein R₄ is H, allyl carbamate, or mixtures thereof; and

(d) optionally adding to the product of step (c) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(e) curing the product of step (c) or step (d) with UV light and/or visible light.

Suitable pore sizes for the cured interstitial EMR-drug compositions or EMR-drug conjugates of the invention are about 0.001 microns to about 100 microns, about 5 microns to about 90 microns, about 10 microns to about 80 microns, about 15 microns to about 70 microns, about 20 microns to about 60 microns, or about 25 microns to about 50 microns.

This invention provides a pharmaceutical composition comprising the interstitial EMR-drug composition or the EMR-drug conjugate of the invention and at least one pharmaceutically acceptable excipient.

This invention provides a method of treating wounds, comprising applying to a wound in a patient in need thereof an effective amount of the interstitial EMR-drug composition or the EMR-drug conjugate of the invention. An effective amount of the interstitial EMR-drug composition or the EMR-drug conjugate is an amount such that wound healing occurs faster for wounds treated with the interstitial EMR-drug composition or the EMR-drug conjugate than occurs for a control, e.g., an untreated wound or a wound treated with the EMR not conjugated to the drug. A patient in need thereof includes e.g., a mammal having a wound as described herein. The mammal may be, e.g., a primate, e.g., a human or a monkey, a horse, a cow, a pig, a dog, a cat, or a mouse.

Another embodiment of the invention is a method of treating wounds in a subject in need thereof with an effective amount of an interstitial EMR-drug composition or an EMR-drug conjugate of this invention, wherein the effective amount of the interstitial EMR-drug composition or the EMR-drug conjugate is an amount such that wound healing occurs faster in wounds treated with the interstitial EMR-drug composition or the EMR-drug conjugate, than occurs in a control, e.g. an untreated wound or a wound treated with the EMR not conjugated to the drug.

Another embodiment of the invention is a method of treating wounds with an effective amount of the interstitial EMR-drug composition or the EMR-drug conjugate s of this invention, wherein wound healing occurs within between 1 day and 100 days after applying the interstitial EMR-drug composition or the EMR-drug conjugate s to the wound, or between 1 day and 10 days after applying the interstitial EMR-drug composition or the EMR-drug conjugate s to the wound.

Another embodiment of the invention is a method of treating wounds with an effective amount of the interstitial EMR-drug composition or the EMR-drug conjugate of this invention, wherein the interstitial EMR-drug composition or the EMR-drug conjugate is applied twice daily, once daily, twice weekly, once weekly, twice monthly, or once monthly.

Another embodiment of the invention is a method of treating wounds with an effective amount of the interstitial EMR-drug composition or the EMR-drug conjugate of this invention, wherein the wounds are acute wounds or chronic wounds.

Another embodiment of the invention is a method of treating wounds with an effective amount of the interstitial EMR-drug composition or the EMR-drug conjugate of this invention, wherein the wounds are excision wounds or burn wounds.

Another embodiment of the invention is a method of treating wounds with an effective amount of the interstitial EMR-drug composition or the EMR-drug conjugate of this invention, wherein the wounds are diabetic ulcers or pressure wounds.

Another embodiment of the invention is a method of treating wounds with an effective amount of the interstitial EMR-drug composition or the EMR-drug conjugate of this invention, wherein the interstitial EMR-drug composition or the EMR-drug conjugate is applied to the wound and subsequently degraded by endogenous enzyme activity in the wound bed as healing proceeds.

Another embodiment of the invention is a method of delivering a drug to a wound in a subject in need thereof, comprising applying an effective amount of the interstitial EMR-drug composition or the EMR-drug conjugate of this invention. The effective amount of the interstitial EMR-drug composition or the EMR-drug conjugate of this invention is an amount such that wound healing occurs faster in wounds treated with the interstitial EMR-drug composition or the EMR-drug conjugate, than occurs in a control, e.g. an untreated wound or a wound treated with the EMR not conjugated to the drug.

Another embodiment of the invention is a method of prolonging delivery of a drug to a wound in a subject in need thereof, comprising applying an effective amount of the interstitial EMR-drug composition or the EMR-drug conjugate of this invention, wherein the interstitial EMR-drug composition or the EMR-drug conjugate is applied twice daily, once daily, twice weekly, once weekly, twice monthly, or once monthly, and wherein delivery of the drug occurs over at least 12 hours, at least 24 hours, at least 7 days, at least 15 days, or at least 30 days.

In the methods of this invention the interstitial EMR-drug composition or the EMR-drug conjugate of this invention may be applied to the wound in the form of a pharmaceutical composition comprising the interstitial EMR-drug composition or the EMR-drug conjugate and a pharmaceutically acceptable excipient.

### EMRs Comprising Fluorescent Labels

Another embodiment of the invention is an EMR combined with a fluorescent label. THE EMRs comprising a fluorescent label as described herein may be applied to a wound (e.g., a chronic wound, such as a diabetic ulcer).

In another embodiment of the invention, an EMR is combined with a fluorescent label to indirectly monitor the progression of wound healing. The EMR comprising the fluorescent label is applied to the wound, e.g., a chronic wound, such as a diabetic ulcer, and the level of fluorescence is measured over time. By measuring the level of fluorescence of the EMR and the surrounding wound environment over time one measures indirectly the progress of wound healing as the EMR degrades and the fluorescent label is released. One may also assess the release of a drug from an EMR-drug conjugate or interstitial EMR-drug composition by combining such EMRs with an EMR-fluorescent label conjugate, applying the EMRs to the wound, and measuring a change in fluorescence in the EMR-fluorescent label conjugate or the surrounding wound environment, which change reflects a degradation of the EMR and the associated release of the drug into the wound.

In another embodiment of the invention, an EMR is combined with fluorescent labels that facilitate assessment of degradation rate or products of the EMR. The method comprises combining an EMR with a fluorescent label and then measuring the level of fluorescence in the EMR, and/or the surrounding environment over time. Preferably the fluorescent label is conjugated to a polymerizable entity of the EMR. As the EMR is degraded the fluorescent label is released and the fluorescence in the EMR and/or in the surrounding environment changes. Those of skill in the art appreciate that depending on the fluorescent label incorporated into the EMR, the intensity or amount of the fluorescence will decrease or be quenched when it is released from the EMR or the intensity or amount of fluorescence will increase once it is released from the EMR or the fluorescence wavelength will change when it is released from the EMR. The fluorescent label may be conjugated to a polymerizable entity or to an acrylate-comprising compound as described herein or by any other suitable conjugation method. The fluorescent label may also be trapped in the interstices of the polymers of the cured EMR.

In an embodiment of this invention, the fluorescent labels are selected from dyes that have emission wavelengths in the range of 350 to 2500 nm. In a preferred embodiment, the fluorescent labels are selected from dyes that have emission wavelengths in the range of 350 to 780 nm. In another preferred embodiment, the fluorescent labels are selected from dyes that have emission wavelengths in the range of 350 to 650 nm. Suitable dyes include CF®350, CF®4055, CF®405M, CF®405L, CF®430, CF®440, CF®450, CF®488A, CF®514, CF®532, CF®535ST, CF®543, CF®555, CF®568, CF®570, CF®583, CF®594, CF®594ST, CF®620R, CF®633, CF®640R, CF®647, CF®660C, CF®660R, CF®680, CF®680R, near-infrared CF® dyes, CF® dyes for multi-color super-resolution microscopy, and mixtures thereof. Suitable dyes also include fluorescein, 5-fluoresceinamine, rhodamine, acridine yellow, and mixtures thereof.

In another embodiment, EMR-fluorescent label conjugates of the invention closely match the physical properties of unfunctionalized EMRs, including the swelling properties, the stiffness, the porosity, and the oxygen permeability. The properties of the EMR-fluorescent label conjugates, like the properties of unfunctionalized EMRs, are determined through conventional methods, as described above for the interstitial EMR-drug compositions or EMR-drug conjugates. For example, a suitable Young's modulus for the EMR-fluorescent label conjugates of the invention is between about 10 Pa and about 2000 Pa, between about 10 Pa and about 1500 Pa, between about 10 Pa and about 1000 Pa, between about 10 Pa and about 500 Pa, and between about 10 Pa and about 250 Pa. Suitable oxygen permeability for the EMR-fluorescent label conjugates of the invention is full oxygen permeability under normoxic conditions.

The swelling ratios of the EMR-fluorescent label conjugates are determined via gravimetric analysis, as described above for interstitial EMR-drug compositions or EMR-drug conjugates to evaluate the capacity of the EMR-fluorescent label conjugates to absorb water (as a surrogate for wound exudate) and desorb water (as a surrogate for wound hydration).

As described above for the interstitial EMR-drug compositions or the EMR-drug conjugates, standard mechanical measurements of the elastic modulus are collected for all EMR-fluorescent label conjugates samples using a rheometer.

As described above for the interstitial EMR-drug compositions or EMR-drug conjugates, the morphology and porosity of all EMR-fluorescent label conjugates can be determined using scanning electron microscopy to provide a representative view of the EMR-fluorescent label conjugate microstructure.

The EMR-fluorescent label conjugates must be oxygen permeable to ensure wound healing and prevent anaerobic bacterial infections. The rate of oxygen transfer through hydrated EMR-fluorescent label conjugates of various thicknesses is measured continuously using oxygen sensors under both physiological and hypoxic conditions. Monitoring oxygen permeability over time provides a temporal profile of oxygen transfer through the EMR.

In all of these characterization steps, the EMR-fluorescent label conjugates of the invention are compared to unfunctionalized EMR as a control, as described above for the interstitial EMR-drug compositions or EMR-drug conjugates.

Cytotoxicity of the EMR-fluorescent label conjugates may be evaluated by culturing human fibroblasts with the EMR-fluorescent label conjugates of the invention in wells and then quantifying fibroblast viability, morphology, and proliferation in the presence of the EMR-fluorescent label conjugates of the invention.

Cell morphology, fibroblast viability, and proliferation may be analyzed by any well-known method in the art. Fibroblast proliferation may be measured using a WST assay.

As described above for the interstitial EMR-drug compositions or EMR-drug conjugates, a scratch wound assay may be used to evaluate basic fibroblast cell recruitment induced by EMR-fluorescent label conjugates.

In vitro degradation of the EMR in the conjugates of this invention is quantified to determine a suitable degradation time, such that in vivo the EMR stays intact for long enough to provide sufficient mechanical support for cells to migrate into the wound bed but degrades sufficiently to allow further cell invasion and tissue regeneration. Degradation of the EMR may be evaluated as described above for the interstitial EMR-drug compositions or EMR-drug conjugates.

This invention provides an EMR-fluorescent label conjugate of formula (XI):

W-J-Y (XI),

wherein W is a fluorescent label, J is a linker group, and Y is an EMR.

One embodiment of the invention is an EMR-fluorescent label conjugate of formula (XI) wherein the fluorescent label is at least one dye with emission wavelengths in the range of 350 to 2500 nm.

Another embodiment of the invention is an EMR-fluorescent label conjugate of formula (XI) wherein the linker group is

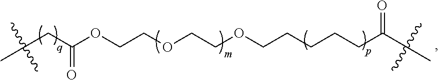

wherein q is 0 or an integer between 1 and 10, m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10.

Another embodiment of the invention is an EMR-fluorescent label conjugate of formula (XI) wherein the linker group is

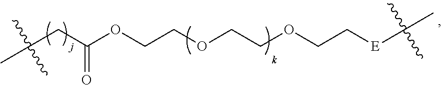

wherein E is O, N, or S, wherein j is 0 or an integer between 1 and 10, and k is 0 or an integer between 1 and 500, between 1 and 250, between 1 and 200, between 1 and 100, between 1 and 50, or between 1 and 10.

Another embodiment of the invention is an EMR-fluorescent label conjugate of formula (XI) wherein the EMR is prepared by curing a compound selected from the group consisting of unfunctionalized glucan, functionalized glucan, and mixtures thereof.

Suitable unfunctionalized glucans that may be used in the EMR-fluorescent label conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the EMR-fluorescent label of the invention, the polymerizable entity is unfunctionalized dextran. Suitable unfunctionalized dextrans that may be used in the EMR-fluorescent label conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the EMR-fluorescent label conjugates of the invention, the polymerizable entity is a functionalized glucan. Suitable functionalized glucans are glucans that are functionalized with polymerizable compounds, such as ethylamine, allyl carbamate, and mixtures thereof. Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the EMR-fluorescent label conjugates of the invention, suitable functionalized glucans have a degree of substitution between about 0.01 and about 0.9, between about 0.02 and about 0.8, between about 0.05 and about 0.7, between about 0.1 and about 0.6, between about 0.15 and about 0.5.

In one embodiment of the method for preparing the EMR-fluorescent label conjugates of the invention, the polymerizable entity is a functionalized dextran. Suitable functionalized dextrans are dextrans that are functionalized with polymerizable compounds, such as ethylamine, allyl carbamate, and mixtures thereof. Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the EMR-fluorescent label conjugates of the invention, suitable functionalized dextrans have a degree of substitution between about 0.01 and about 0.9, between about 0.02 and about 0.8, between about 0.05 and about 0.7, between about 0.1 and about 0.6, between about 0.15 and about 0.5.

In a preferred embodiment of the EMR-fluorescent label conjugates of the invention, the polymerizable entity is dextramate. In some embodiments of the invention, the degree of substitution in the dextramate is between about 0.01 and about 0.9, between about 0.02 and about 0.8, between about 0.05 and about 0.7, between about 0.1 and about 0.6, or between about 0.15 and about 0.5.

For use in the EMR-fluorescent label conjugates of the invention, suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da.

One embodiment of the invention is an EMR-fluorescent label conjugates wherein the EMR component comprises a mixture of a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One preferred embodiment of the invention is an EMR-fluorescent label conjugates wherein the EMR component comprises a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One embodiment of the invention is an EMR-fluorescent label conjugates wherein the fluorescent label component makes up about 0.001% to about 25% of the total weight of the EMR-fluorescent label conjugate, about 0.01% to about 10% of the total weight of the EMR-fluorescent label conjugate, or about 0.1% to about 5% of the total weight of the EMR-fluorescent label conjugate.

Another embodiment of the invention is an EMR-fluorescent label conjugate of formula (XI) wherein the fluorescent label is 5-fluoresceinamine.

This invention provides a method for preparing the EMR-fluorescent label conjugate of the invention, comprising:

(a) reacting a fluorescent label of formula (XI-a):

W₁—H (XI-a)

with a structure of formula (XIIa):

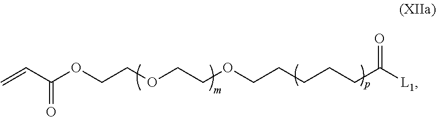

wherein L₁ is selected from the group consisting of —Cl, —Br, —I, and —ORₐ, wherein Rₐ is H, C₁-C₁₀ alkyl,

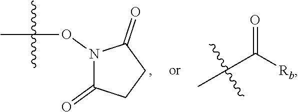

and wherein R_{b} is C₁-C₁₀ alkyl; m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10,

to yield a compound of formula (XIIIa):

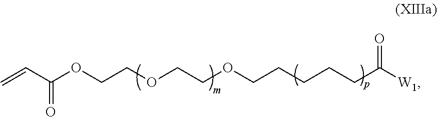

wherein W₁ is selected from the group consisting of:

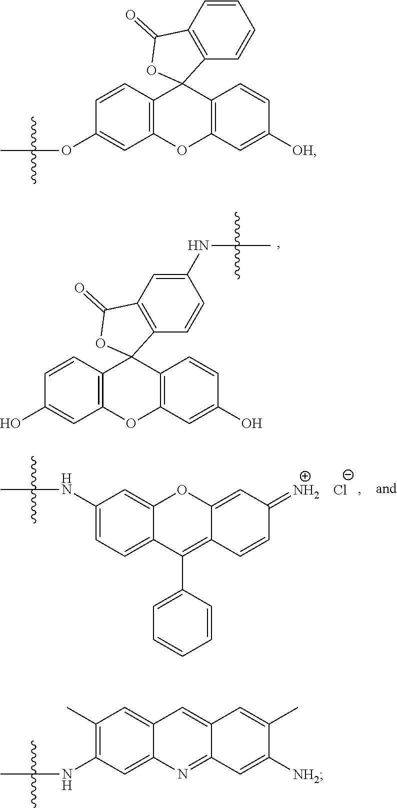 [SMILES: *Oc1ccc2c(c1)Oc1cc(O)ccc1C21OC(=O)c2ccccc21 ; *Nc1ccc2c(c1)C(=O)OC21c2ccc(O)cc2Oc2cc(O)ccc21 ; *Nc1ccc2c(-c3ccccc3)c3ccc(=[NH+2])cc-3oc2c1 ; [Cl-] ; *Nc1cc2nc3cc(N)c(C)cc3cc2cc1C]

m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10;

(b) mixing the compound of formula (XIIIa) with a compound of formula (X):

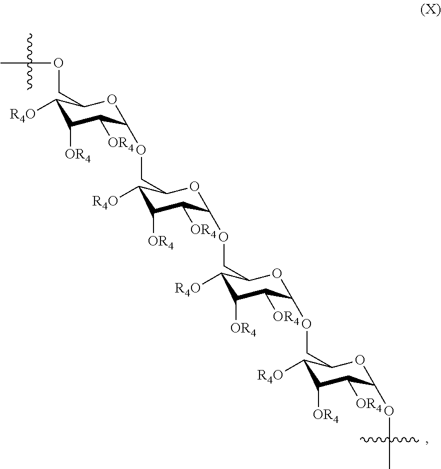

wherein R₄ is H, allyl carbamate, or mixtures thereof;

(c) optionally mixing an acrylate-comprising compound with the product of step (b); and

(d) curing the product of step (b) or step (c) with UV light and/or visible light.

Another embodiment of the invention is a method for preparing the EMR-fluorescent label conjugate of the invention, comprising:

(a) reacting a fluorescent label of formula (XI-a):

W₁—H (XI-a)

with a structure of formula (XVI):

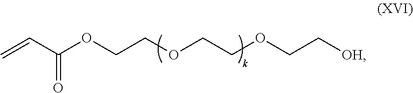

wherein k is 0 or an integer between 1 and 500, between 1 and 250, between 1 and 200, between 1 and 100, between 1 and 50, or between 1 and 10,

to yield a compound of formula (XVII):

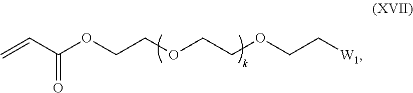

wherein W₁ is selected from the group consisting of:

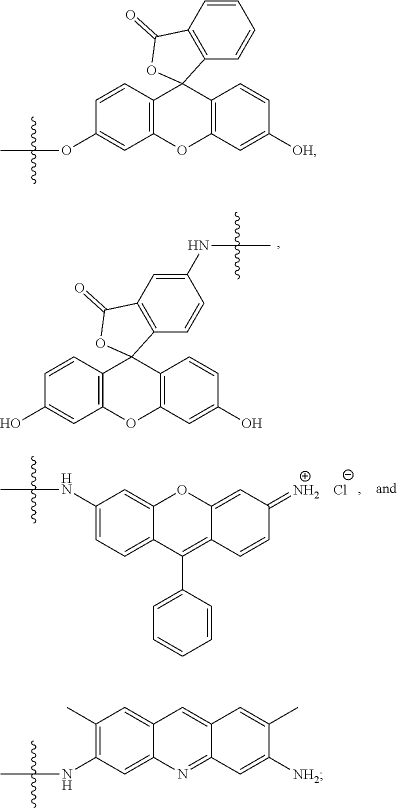 [SMILES: *Oc1ccc2c(c1)Oc1cc(O)ccc1C21OC(=O)c2ccccc21 ; *Nc1ccc2c(c1)C(=O)OC21c2ccc(O)cc2Oc2cc(O)ccc21 ; *Nc1ccc2c(-c3ccccc3)c3ccc(=[NH+2])cc-3oc2c1 ; [Cl-] ; *Nc1cc2nc3cc(N)c(C)cc3cc2cc1C]

and

wherein k is 0 or an integer between 1 and 500, between 1 and 250, between 1 and 200, between 1 and 100, between 1 and 50, or between 1 and 10;

(b) mixing the compound of formula (XVII) with a compound of formula (X):

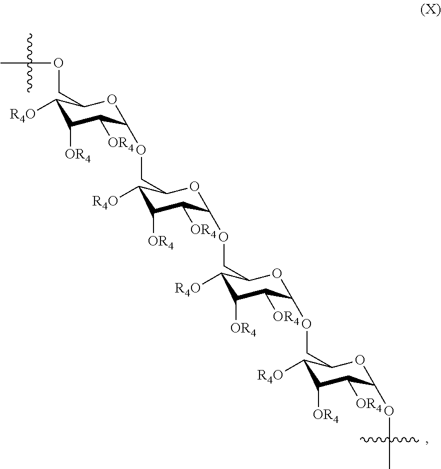

wherein R₄ is H, allyl carbamate, or mixtures thereof;

(c) optionally mixing an acrylate-comprising compound with the product of step (b); and

(d) curing the product of step (b) or step (c) with UV light and/or visible light.

In another embodiment, the EMR-fluorescent label conjugate of the invention is prepared by a method comprising the steps of:

(a) mixing a compound of formula (X):

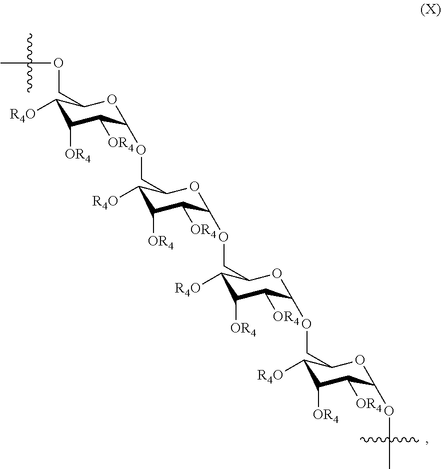

wherein R₄ is H, allyl carbamate, or mixtures thereof,

with an acrylate-comprising compound selected from the group consisting of polyethylene(glycol)diacrylate, acrylate-polyethylene(glycol)-succinimidyl valeric acid, polyethylene(glycol)acrylate, and mixtures thereof;

(b) curing the product of step (a) with UV light and/or visible light; and

(c) reacting the product of step (b) with a fluorescent label of formula (XI-a):

W₁—H (XI-a),

wherein W₁ is selected from the group consisting of:

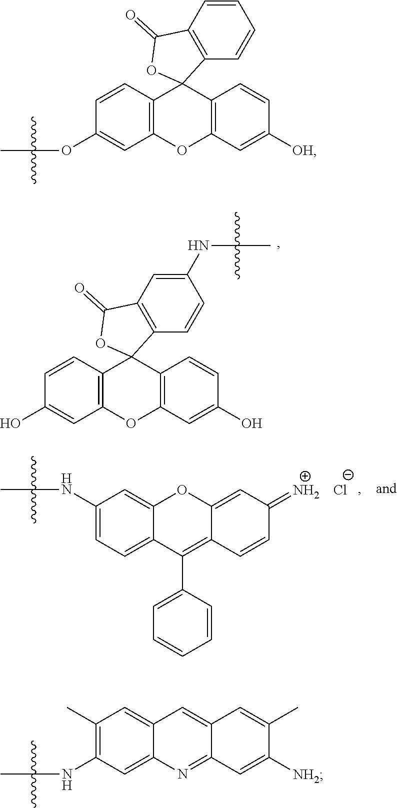 [SMILES: *Oc1ccc2c(c1)Oc1cc(O)ccc1C21OC(=O)c2ccccc21 ; *Nc1ccc2c(c1)C(=O)OC21c2ccc(O)cc2Oc2cc(O)ccc21 ; *Nc1ccc2c(-c3ccccc3)c3ccc(=[NH+2])cc-3oc2c1 ; [Cl-] ; *Nc1cc2nc3cc(N)c(C)cc3cc2cc1C]

In another embodiment, the EMR-fluorescent label conjugate of the invention is prepared by a method comprising the steps of:

(a) mixing a compound of formula (X):

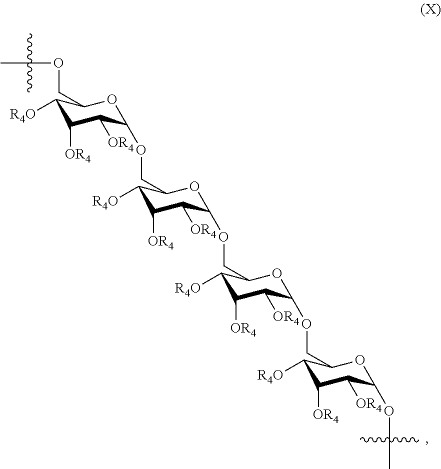

wherein R₄ is H, allyl carbamate, or mixtures thereof,

with a compound selected from the group consisting of polyethylene(glycol)diacrylate, acrylate-polyethylene(glycol)-succinimidyl valeric acid, polyethylene(glycol)acrylate, and mixtures thereof;

(b) reacting the product of step (a) with a fluorescent label of formula (XI-a):

W₁—H (XI-a),

wherein W₁ is selected from the group consisting of:

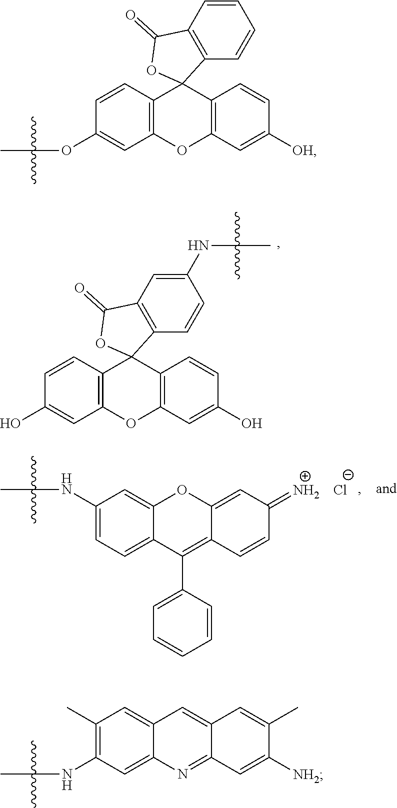 [SMILES: *Oc1ccc2c(c1)Oc1cc(O)ccc1C21OC(=O)c2ccccc21 ; *Nc1ccc2c(c1)C(=O)OC21c2ccc(O)cc2Oc2cc(O)ccc21 ; *Nc1ccc2c(-c3ccccc3)c3ccc(=[NH+2])cc-3oc2c1 ; [Cl-] ; *Nc1cc2nc3cc(N)c(C)cc3cc2cc1C]

and

(c) curing the product of step (b) with UV light and/or visible light.

Suitable pore sizes for the EMR-fluorescent label conjugates of the invention are about 0.001 microns to about 100 microns, about 5 microns to about 90 microns, about 10 microns to about 80 microns, about 15 microns to about 70 microns, about 20 microns to about 60 microns, or about 25 microns to about 50 microns.

This invention provides a pharmaceutical composition comprising the EMR-fluorescent label conjugate of the invention and at least one pharmaceutically acceptable excipient.

This invention also provides a method of measuring the progress of wound treatment, comprising applying an EMR-fluorescent label conjugate to a wound and measuring healing of the wound via fluorescence microscopy and/or fluorescent spectrophotometer. Measuring may occur twice a day, once a day, twice a week, once a week, twice a month, or once a month.

This invention also provides a method of indirectly measuring the amount and/or rate of drug release from an EMR-drug conjugate, comprising applying an EMR-fluorescent label conjugate and an EMR-drug conjugate to a wound and measuring the amount and/or rate of fluorescence released from the EMR-fluorescent conjugate. The amount of fluorescence in the EMR and the surrounding wound environment may be measured via fluorescence microscopy and/or fluorescent spectrophotometer over time. The amount of fluorescence in the EMR and the surrounding wound environment is indicative of the level of EMR degradation and thus an indirect measure of drug release from the EMR-drug conjugate or EMR interstitial drug composition. Measuring may occur twice a day, once a day, twice a week, once a week, twice a month, or once a month.

This invention also provides a method of indirectly measuring the degradation products of an EMR, comprising applying an EMR-fluorescent label conjugate to a wound and measuring the amount of fluorescence in the EMR and the surrounding wound environment. The amount of fluorescence in the EMR-fluorescent composition and the surrounding wound environment is indicative of the amount of degradation products of the EMR. The amount of fluorescent label in the EMR and surrounding environment may be measured via fluorescence microscopy and/or fluorescent spectrophotometer over time. Measuring may occur twice a day, once a day, twice a week, once a week, twice a month, or once a month.

This invention also provides a method of measuring the progress of wound treatment, comprising applying an EMR-fluorescent label conjugate to a wound and measuring amount of fluorescence in the EMR and the surrounding wound environment. The amount of fluorescence in the EMR-fluorescent composition and the surrounding wound environment is indicative of the amount of healing of the wound. The amount of fluorescence in the EMR-fluorescent composition and the surrounding wound environment may be measured via fluorescence microscopy and/or fluorescent spectrophotometer. Measuring may occur twice a day, once a day, twice a week, once a week, twice a month, or once a month.

In the methods of this invention the EMR-fluorescent label conjugate of this invention may be applied to the wound in the form of a pharmaceutical composition comprising the EMR-fluorescent label conjugate and a pharmaceutically acceptable excipient.

### Cure-in-Place Extracellular Matrix Replacements

As previously described, EMRs, EMR-drug conjugates, and EMR-fluorescent label conjugates may be generated by mixing one or more polymerizable entities and curing using UV light and/or visible light. Once the EMR, EMR-drug conjugate, or EMR-fluorescent label conjugate is generated, it is swelled in water, packaged, and sterilized. Clinicians may then apply the EMR, EMR-drug conjugate, or EMR-fluorescent label conjugate as a patch by cutting it to size and placing it directly in the wound bed.

An embodiment of this invention is a cure-in-place (CIP) EMR. These CIP-EMRs allow a wound-specific fit by filling the wound with uncured or partially cured CIP-EMRs having a high viscosity and then curing the uncured or partially cured CIP-EMRs in the filled wound with UV light and/or visible light. Treatment of chronic wounds with the CIP-EMRs improves healing, reduces scarring, and reduces wound dehiscence for a variety of wounds.

The CIP-EMRs described herein fully fill an irregularly-shaped wound to give maximum contact between the CIP-EMRs and the wound bed. Without wishing to be bound by theory, it is contemplated that the CIP-EMRs improve healing in at least two ways: (1) by mechanically stabilizing the entire wound bed; and/or (2) by stimulating cell migration and tissue regeneration as a result of the formulation having a more complete contact with the wound bed.

In an embodiment of this invention, the CIP-EMRs of the invention comprise a high-viscosity solution comprising at least one functionalized glucan (e.g., at least one functionalized dextran), and/or at least one acrylate-comprising compound, and/or at least one substituted glucan (e.g., at least one substituted dextran), and/or at least one functionalized acrylate-comprising compound.

Examples of suitable functionalized glucans are glucans functionalized with e.g., ethylamine, allyl carbamate, or mixtures thereof. Examples of suitable acrylate-comprising compound are polyethylene(glycol)diacrylate, acrylate-polyethylene(glycol)-succinimidyl valeric acid, polyethylene(glycol)acrylate, or mixtures thereof.

Examples of suitable substituted glucans are glucans functionalized with e.g., small molecules comprising a carboxylic acid (e.g., ARBs, antibiotics, and/or analgesics) and/or functionalized with fluorescent labels (e.g., 5-fluorescein amine).

Examples of suitable functionalized acrylate-comprising compounds are acrylate-comprising compounds (e.g., polyethylene(glycol)diacrylate, acrylate-polyethylene(glycol)-succinimidyl valeric acid, polyethylene(glycol)acrylate, or mixtures thereof) that are functionalized with small molecules comprising a carboxylic acid (e.g., ARBs, antibiotics, analgesics), fluorescent labels (e.g., 5-fluorescein amine), or mixtures thereof.

For use in the CIP-EMRs of the invention, the same glucans described above for the interstitial EMR-drug compositions and EMR-drug conjugates of the invention may be used.

Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable functionalized glucans have a degree of substitution between about 0.01 and about 0.9, between about 0.02 and about 0.8, between about 0.05 and about 0.7, between about 0.1 and about 0.6, between about 0.15 and about 0.5.

Suitable molecular weight ranges for substituted glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable substituted glucans have a degree of substitution between about 0.01 and about 0.9, between about 0.02 and about 0.8, between about 0.05 and about 0.7, between about 0.1 and about 0.6, between about 0.15 and about 0.5.

For use in the CIP-EMRs of the invention, the same dextrans described above for the interstitial EMR-drug compositions and EMR-drug conjugates of the invention may be used.

Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable functionalized dextrans have a degree of substitution between about 0.01 and about 0.9, between about 0.02 and about 0.8, between about 0.05 and about 0.7, between about 0.1 and about 0.6, between about 0.15 and about 0.5.

Suitable molecular weight ranges for substituted dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable substituted dextrans have a degree of substitution between about 0.01 and about 0.9, between about 0.02 and about 0.8, between about 0.05 and about 0.7, between about 0.1 and about 0.6, between about 0.15 and about 0.5.

For use in the CIP-EMRs of the invention, the same acrylate-comprising compounds described above for the interstitial EMR-drug compositions and EMR-drug conjugates of the invention may be used.

Suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da.

Suitable functionalized acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da.

A preferred embodiment of the CIP-EMRs of the invention comprises a high-viscosity solution comprising a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

Another preferred embodiment of the CIP-EMRs of the invention comprises a high-viscosity solution comprising a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

In another embodiment of this invention, the CIP-EMRs of the invention further comprise one or more UV-crosslinking catalysts. Examples of UV-crosslinking catalysts include Irgacure catalysts (e.g., Irgacure 2959) and lithium phenyl-2,4,6-trimethylbenzoylphosphinate (LAP).

In another embodiment of this invention, the CIP-EMRs of the invention further comprise cross-linking catalysts that catalyze curing in the visible spectrum (i.e., in wavelengths ranging from 390 to 700 nm). Examples of visible-light cross-linking catalysts include eosin-Y.

The invention provides methods for preparing CIP-EMRs, comprising the steps of preparing a low-viscosity solution comprising at least one functionalized dextran and converting the low-viscosity solution into a high-viscosity solution. The low-viscosity solution may further comprise at least one acrylate-comprising compound and/or a UV-crosslinking catalyst and/or a visible light-crosslinking catalyst.

Examples of high-viscosity solutions include solutions comprising at least 10% (w/w) functionalized dextran and/or substituted dextran; at least 20% (w/w) functionalized dextran and/or substituted dextran, at least 30% (w/w) functionalized dextran and/or substituted dextran, at least 40% (w/w) functionalized dextran and/or substituted dextran, and at least 50% (w/w) functionalized dextran and/or substituted dextran. The high viscosity solutions may further comprise an acrylate-comprising compound or a UV-crosslinking catalyst or visible light-crosslinking catalyst or combinations of an acrylate-comprising compound, a UV-crosslinking catalyst and/or a visible light-crosslinking catalyst. For example, the high-viscosity solutions may further comprise 10% (w/w) acrylate-comprising compound and/or functionalized acrylate-comprising compound, at least 20% (w/w) acrylate-comprising compound and/or functionalized acrylate-comprising compound, at least 30% (w/w) acrylate-comprising compound and/or functionalized acrylate-comprising compound, at least 40% (w/w) acrylate-comprising compound and/or functionalized acrylate-comprising compound, and at least 50% (w/w) acrylate-comprising compound and/or functionalized acrylate-comprising compound. The high-viscosity solutions of this invention may further comprise at least 1% (w/w) UV-crosslinking catalysts and/or visible light-crosslinking catalysts, at least 2% (w/w) UV-crosslinking catalysts and/or visible light-crosslinking catalysts, at least 3% (w/w) UV-crosslinking catalysts and/or visible light-crosslinking catalysts, at least 4% (w/w) UV-crosslinking catalysts and/or visible light-crosslinking catalysts, and at least 5% (w/w) UV-crosslinking catalysts and/or visible light-crosslinking catalysts.

Examples of low-viscosity solutions include solutions comprising up to 10% (w/w) functionalized and/or substituted dextran; up to 9% (w/w) functionalized and/or substituted dextran, up to 8% (w/w) functionalized and/or substituted dextran, up to 7.5% (w/w) functionalized and/or substituted dextran, and up to 5% (w/w) functionalized and/or substituted dextran. The low viscosity solutions may further comprise an acrylate-comprising compound or a UV-crosslinking catalysts and/or visible light-crosslinking catalysts or combinations of the an acrylate-comprising compound or a UV-crosslinking catalysts and/or visible light-crosslinking catalysts. For example, the low-viscosity solutions may include up to 10% (w/w) acrylate-comprising compound and/or functionalized acrylate-comprising compound, up to 9% (w/w) acrylate-comprising compound and/or functionalized acrylate-comprising compound, up to 8% (w/w) acrylate-comprising compound and/or functionalized acrylate-comprising compound, up to 7.5% (w/w) acrylate-comprising compound and/or functionalized acrylate-comprising compound, and up to 5% (w/w) acrylate-comprising compound and/or functionalized acrylate-comprising compound. The low-viscosity solutions may further comprise up to 1% (w/w) UV-crosslinking catalysts and/or visible light-crosslinking catalysts, up to 0.9% (w/w) UV-crosslinking catalysts and/or visible light-crosslinking catalysts, up to 0.8% (w/w) UV-crosslinking catalysts and/or visible light-crosslinking catalysts, up to 0.75% (w/w) UV-crosslinking catalysts and/or visible light-crosslinking catalysts, and up to 0.5% (w/w) UV-crosslinking catalysts and/or visible light-crosslinking catalysts.

Further examples of low-viscosity solutions include solutions comprising 8% allyl carbamate-dextran (dextramate)/2% PEGDA/0.1% Irgacure 2959 (w/w). Viscosity may be determined using a viscometer. Further examples of low-viscosity solutions include solutions with a room-temperature viscosity ranging from 0.0091 poise to 14.12 poise. Examples of high-viscosity solutions are solutions with a room-temperature viscosity higher than 14.12 poise.

In an embodiment of this invention, the low-viscosity solution has a room-temperature viscosity ranging from 0.0091 poise to 14.12 poise, from 0.0091 poise to 12 poise, from 0.0091 poise to 10 poise, from 0.0091 poise to 5 poise, from 0.0091 poise to 2 poise, from 0.0091 poise to 1 poise, from 0.0091 poise to 0.50 poise, from 0.0091 poise to 0.10 poise, from 0.0091 poise to 0.05 poise.

In an embodiment of this invention, the low-viscosity solution is aqueous.

In an embodiment of this invention, the low-viscosity solution is converted into a high-viscosity solution by increasing the concentration of at least one functionalized dextran, and/or at least one substituted dextran, and/or at least one acrylate-comprising compound, and/or at least one functionalized acrylate-comprising compound.

Suitable concentrations may be determined by known methods. For example, the water content of a low-viscosity solution may be titrated down until the solution is saturated with at least one functionalized dextran, and/or at least one substituted dextran, and/or at least one acrylate-comprising compound, and/or at least one functionalized acrylate-comprising compound. An example of high-viscosity solution comprises 5.6 g dextramate, 1.4 g PEGDA, 0.1% Irgacure, and water to bring the solution to 10 mL total.

In an embodiment of this invention, the low-viscosity solution is converted into a high-viscosity solution by adding high-viscosity, non-irritating polar solvents and/or solvent additives. Suitable solvents include, for example, glycerol (1412 cP), medical honey (10,000 cP), and isopropanol (1.96 cP). Suitable solvent additives include, for example, BYK-420 and Garamite-7305. For example, a low viscosity solution comprising 800 mg dextramate, 200 mg PEGDA, and 0.1% Irgacure in 10 mL water may be converted into a high-viscosity solution by adding glycerol and/or replacing some or all of water with glycerol. An example of a high-viscosity solution comprises 800 mg dextramate, 200 mg PEGDA, and 0.1% Irgacure, and 10 g glycerol.

In an embodiment of this invention, the low-viscosity solution is converted into a high-viscosity solution by partially curing the low-viscosity solution with UV-light and/or visible light. Conditions for partially curing the low-viscosity solutions may be determined using known methods. For example, conditions may be determined by modifying the conditions for curing known EMRs and systematically reducing the curing times and/or UV intensity and/or visible light intensity required for curing those known EMRs.

In an embodiment of this invention, solutions are considered to have a suitably high viscosity when they are capable of filling a wound bed, and/or are capable of conforming to wound boundaries, and/or when they are capable of creating a uniformly-cured EMR upon exposure to UV light and/or visible light.

This invention provides a pharmaceutical composition comprising the CIP-EMRs of the invention and at least one pharmaceutically acceptable excipient.

In an embodiment of this invention, a solution comprising at least one functionalized dextran and optionally an acrylate-comprising compound for making the CIP-EMRs of the invention may be packaged in a light-blocking container as a pre-mixed suspension.

In an embodiment of this invention, the CIP-EMRs of the invention further comprise a drug with a carboxylic acid group, e.g., an ARB, conjugated to the EMR. The ARB may be selected from the group consisting of valsartan, olmesartan, azilsartan, eprosartan, candesartan, telmisartan, carboxylosartan, irbesartan, losartan, and mixtures thereof.

In an embodiment of this invention, the CIP-EMRs comprise at least one substituted dextran that is functionalized with a small molecule, and/or at least one acrylate-comprising compound that is functionalized with a small molecule, and/or at least one acrylate-comprising compound. These small molecules in the CIP-EMRs of the invention also accelerate wound closure, prevent wound infection, and/or provide pain relief.

This invention provides substituted dextrans of formula (XXI), which are functionalized with a small molecule comprising a carboxylic acid group, that may be used in the CIP-EMRs of the invention:

Q₃-X—Y₁ (I-1),

wherein Q₃ is the residue of a small molecule comprising a carboxylic acid group selected from the group consisting of ARBs, antibiotics, analgesics, and mixtures thereof;

X is a linker group selected from the group consisting of a bond,

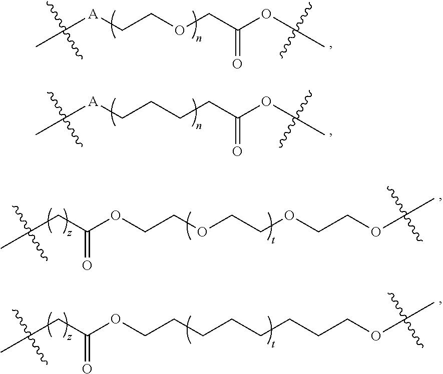

and mixtures thereof, wherein A is O, S, or NH, n is 0 or an integer between 1 and 500, between 1 and 250, between 1 and 200, between 1 and 100, between 1 and 50, or between 1 and 10; t is 0 or an integer between 1 and 500, between 1 and 250, between 1 and 200, between 1 and 100, between 1 and 50, or between 1 and 10; and z is 0 or an integer between 1 and 10; and

Y₁ is a dextran functionalized with ethylamine, allyl carbamate, or mixtures thereof.

In one embodiment, the dextrans of formula (XXI) are prepared by:

(a) reacting a small molecule of formula (XVIII) with a compound of formula (XIXa) or formula (XIXb) to yield a compound of formula (XXa) or (XXb):

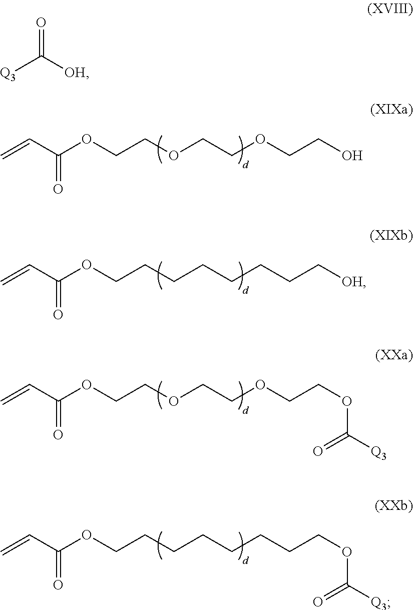

wherein Q₃ is the residue of any small molecule (e.g., a small molecule selected from the group consisting of ARBs, antibiotics, and analgesics) and wherein d is 0 or an integer between 1 and 500, between 1 and 250, between 1 and 200, between 1 and 100, between 1 and 50, or between 1 and 10;

(b) reacting the compound of formula XXa or formula XXb with acryl carbamate groups on a dextran of formula X

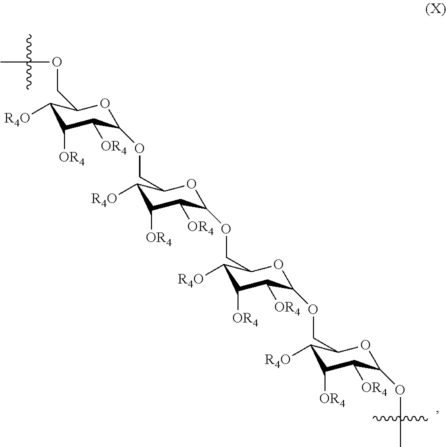

wherein R₄ is H, allyl carbamate, or mixtures thereof.

In one preferred embodiment of the method of making dextrans of formula (XXI), Q₃ is selected from the group consisting of

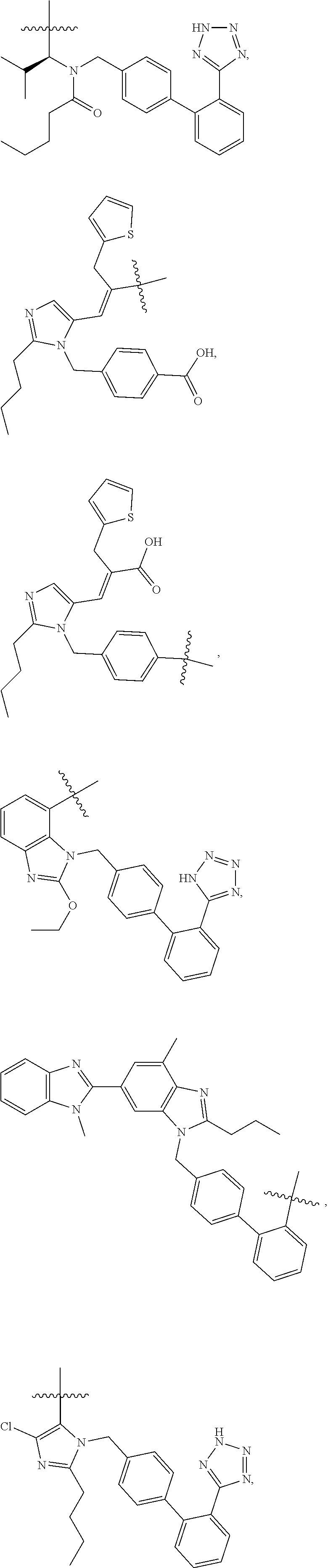

and mixtures thereof.

FIG. 3A illustrates reacting polyethylene(glycol)acrylate (10 mg) with valsartan (8.7 mg) using dicyclohexylcarbodiimide (DCC, 4.95 mg) and dimethylaminopyridine (DMAP, 2.93 mg) in dimethylformamide (DMF, 5 mL). FIG. 4A illustrates curing a mixture of the valsartan-functionalized polyethylene(glycol)acrylate resulting from the reaction of FIG. 3A, polyethylene(glycol)diacrylate, and dextramate to make an EMR-drug conjugate.

In an embodiment of this invention, the CIP-EMRs comprise at least one substituted dextran that is functionalized with an ARB and/or comprise at least one acrylate-comprising compound that is functionalized with an ARB and/or at least one acrylate-comprising compound.

In an embodiment of this invention, CIP-EMRs of the invention comprising an ARB accelerate wound closure.

This invention provides substituted dextrans of formula (I-1), which are functionalized with an ARB, that may be used in the CIP-EMRs of the invention:

Q-X—Y₁ (I-1),

wherein Q is an ARB selected from the group consisting of valsartan, olmesartan, azilsartan, eprosartan, candesartan, telmisartan, carboxylosartan, irbesartan, losartan, and mixtures thereof;

X is a linker group selected from the group consisting of a bond,

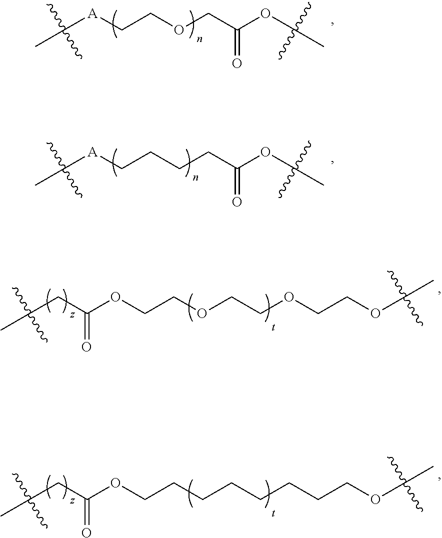

and mixtures thereof, wherein A is O, S, or NH, n is 0 or an integer between 1 and 500, between 1 and 250, between 1 and 200, between 1 and 100, between 1 and 50, or between 1 and 10; t is 0 or an integer between 1 and 500, between 1 and 250, between 1 and 200, between 1 and 100, between 1 and 50, or between 1 and 10; and z is 0 or an integer between 1 and 10; and

Y₁ is a dextran functionalized with ethylamine, allyl carbamate, or mixtures thereof.

This invention provides a method for preparing the substituted dextrans of formula (I-1), which are functionalized with an ARB, comprising:

(a) transforming the ARB with a structure of formula (IIa) into a compound of formula (IIb):

Q₁-OH (IIa)

Q₁-L (IIb),

wherein Q₁ is selected from the group consisting of:

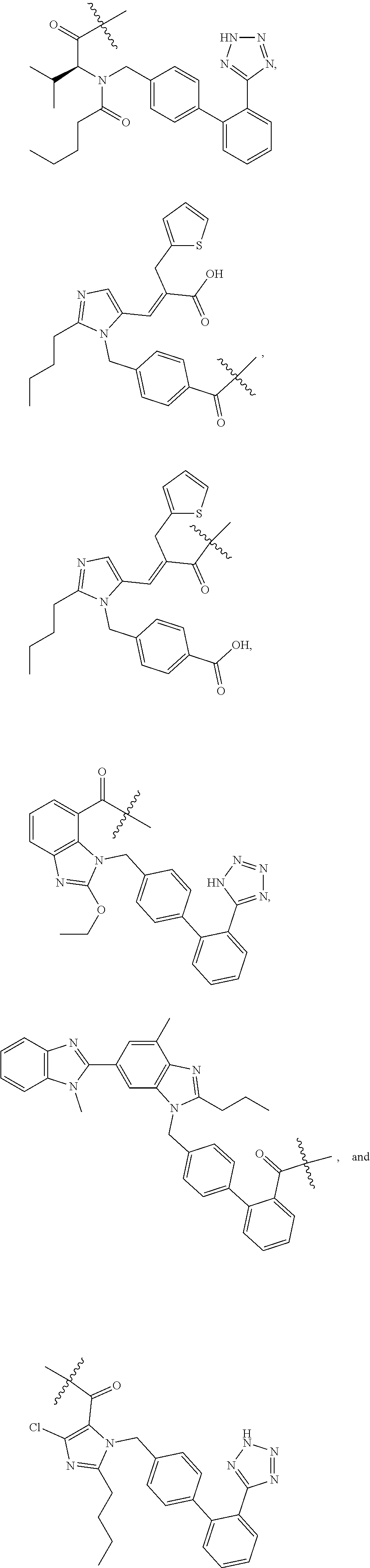

wherein L is selected from the group consisting of: —Cl, —Br, —I, and —OR₁, wherein R₁ is C₁-C₁₀ alkyl or

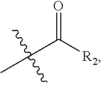

and wherein R₂ is C₁-C₁₀ alkyl;

(b) reacting the compound of formula (IIb) with a compound of formula (IIIa) or formula (IIIb) to yield a compound of formula (IVa) or (IVb):

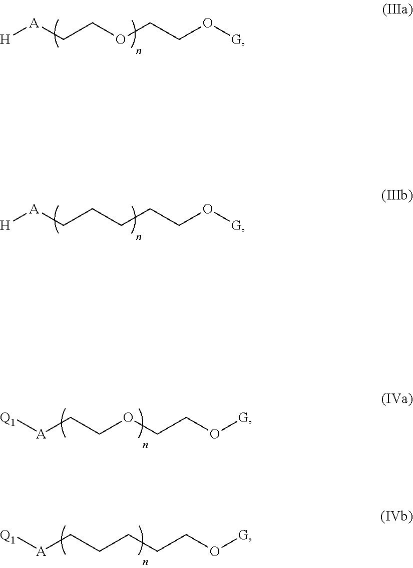

wherein G is a protecting group, A is O, S, or NH, n is 0 or an integer between 1 and 500, between 1 and 250, between 1 and 200, between 1 and 100, between 1 and 50, or between 1 and 10; and Q₁ is the same as defined in step (a);

(c) deprotecting the compound of formula (IVa) or formula (IVb) to yield a compound of formula (Va) or formula (Vb):

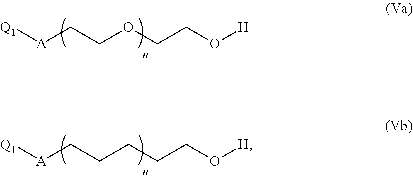

wherein Q₁ is the same as defined in step (a) and A and n are the same as defined in step (b);

(d) oxidizing the compound of formula (IVa) or formula (IVb) to yield a product of formula (VIa) or formula (VIb):

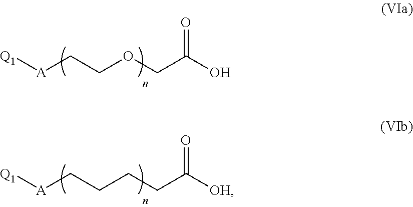

wherein Q₁ is the same as defined in step (a) and A and n are the same as defined in step (b);

(e) transforming the compound of formula (VIa) or formula (VIb) to yield a compound of formula (VIIa) or formula (VIIb):

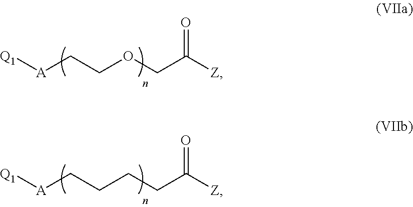

wherein Z is —Cl, —Br, —I, and —OR₃, wherein R₃ is C₁-C₁₀ alkyl, and wherein Q₁ is the same as defined in step (a) and A and n are the same as defined in step (b); and

(f) reacting the product of compound of formula (VIIa) or formula (VIIb) with free hydroxyl groups on a compound of formula (X):

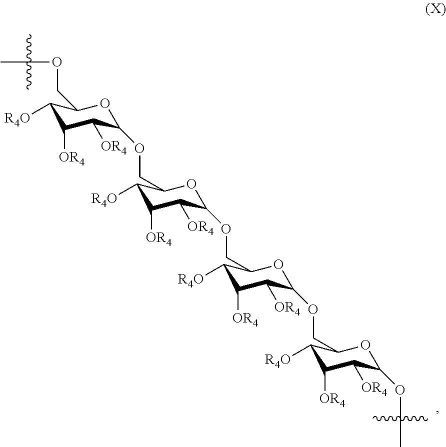

wherein R₄ is H, allyl carbamate, or mixtures thereof.

Another embodiment of the invention is a method for preparing the substituted dextrans of formula (I-1), which are functionalized with an ARB, comprising:

(a) transforming the ARB with a structure of formula (IIc) into a compound of formula (IIa-1):

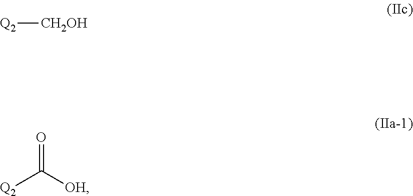

wherein Q₂ is

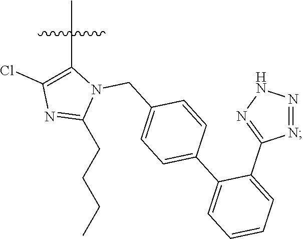 [SMILES: *c1c(Cl)nc(CCCC)n1Cc1ccc(-c2ccccc2-c2nn[nH]n2)cc1]

(b) transforming the compound formula (IIa-1) into a compound of formula (IIb-1):

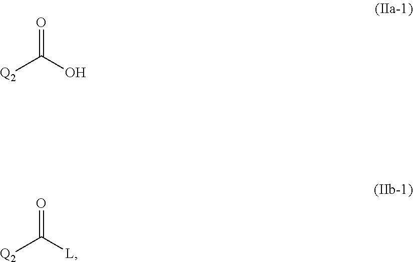

wherein Q₂ is the same as defined in step (a), wherein L is selected from the group consisting of —Cl, —Br, —I, and —OR₁, wherein R₁ is C₁-C₁₀ alkyl or

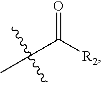

and wherein R₂ is C₁-C₁₀ alkyl;

(c) reacting the compound of formula (IIb-1) with a compound of formula (IIIa) or formula (IIIb) to yield a compound of formula (IVa-1) or (IVb-1):

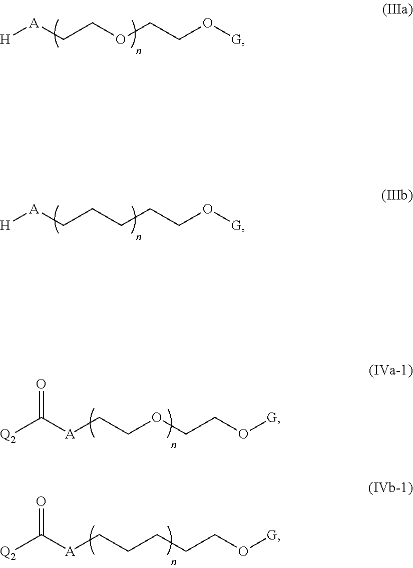

wherein G is a protecting group selected from the group consisting of C₁-C₁₀ unbranched or branched alkyl; —SiMe₃; —SiEt₃; —Si(iPr)₃; —SiPh₃; —SiMe₂iPr; —SiMe₂Et; —SiEt₂iPr; and —CH₂-Ph, wherein the Ph is unsubstituted or substituted with at least one substituent selected from the group consisting of —OMe, —NO₂, —F, —Cl, —Br, —I, —CF₃, —SiMe₃, and —CN,

A is O, S, or NH,

n is 0 or an integer between 1 and 500, between 1 and 250, between 1 and 200, between 1 and 100, between 1 and 50, or between 1 and 10; and Q₂ is the same as defined in step (a);

(d) deprotecting the compound of formula (IVa-1) or formula (IVb-1) to yield a compound of formula (Va-1) or formula (Vb-1):

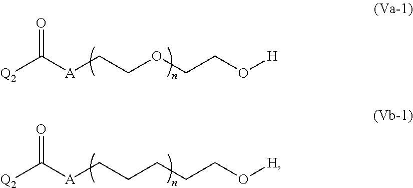

wherein Q₂ is the same as defined in step (a) and A and n are the same as defined in step (c);

(e) oxidizing the compound of formula (IVa-1) or formula (IVb-1) to yield a product of formula (VIa-1) or formula (VIb-1):

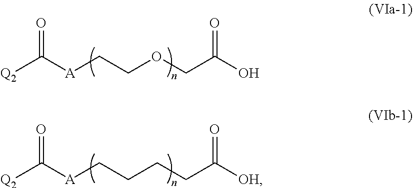

wherein Q₂ is the same as defined in step (a) and A and n are the same as defined in step (c);

(f) transforming the compound of formula (VIa-1) or formula (VIb-1) to yield a compound of formula (VIIa-1) or formula (VIIb-1):

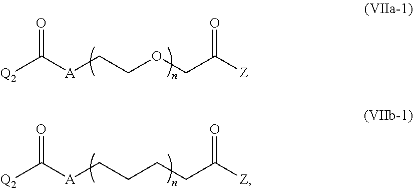

wherein Z is —Cl, —Br, —I, and —OR₃, wherein R₃ is C₁-C₁₀ alkyl, and wherein Q₂ is the same as defined in step (a) and A and n are the same as defined in step (c); and

(g) reacting the product of compound of formula (VIIa-1) or formula (VIIb-1) with free hydroxyl groups on a compound of formula (X):

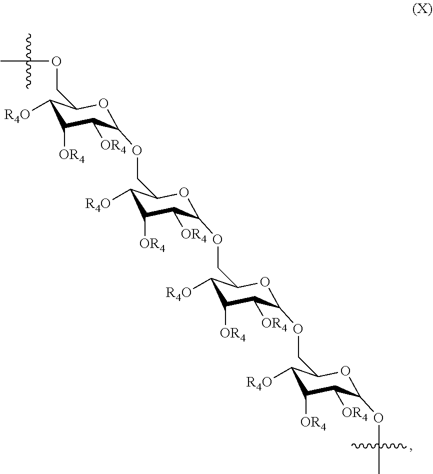

wherein R₄ is H, allyl carbamate, or mixtures thereof.

Another embodiment of the invention is a method for preparing the substituted dextrans of formula (I-1), which are functionalized with an ARB, comprising:

(a) reacting a compound of formula (IIa) with a structure of formula (XIVa) or formula (XIVb) to yield a compound of formula (XVa) or formula (XVb):

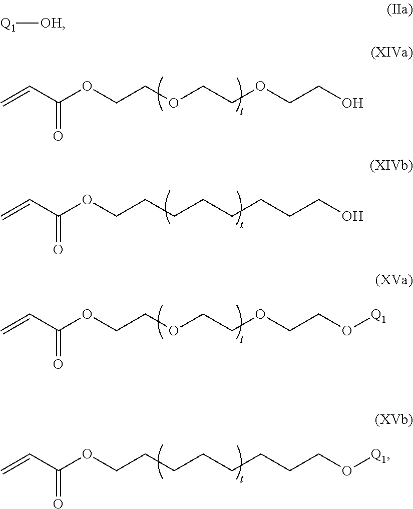

wherein t is 0 or an integer between 1 and 500, between 1 and 250, between 1 and 200, between 1 and 100, between 1 and 50, or between 1 and 10; and Q₁ is selected from the group consisting of:

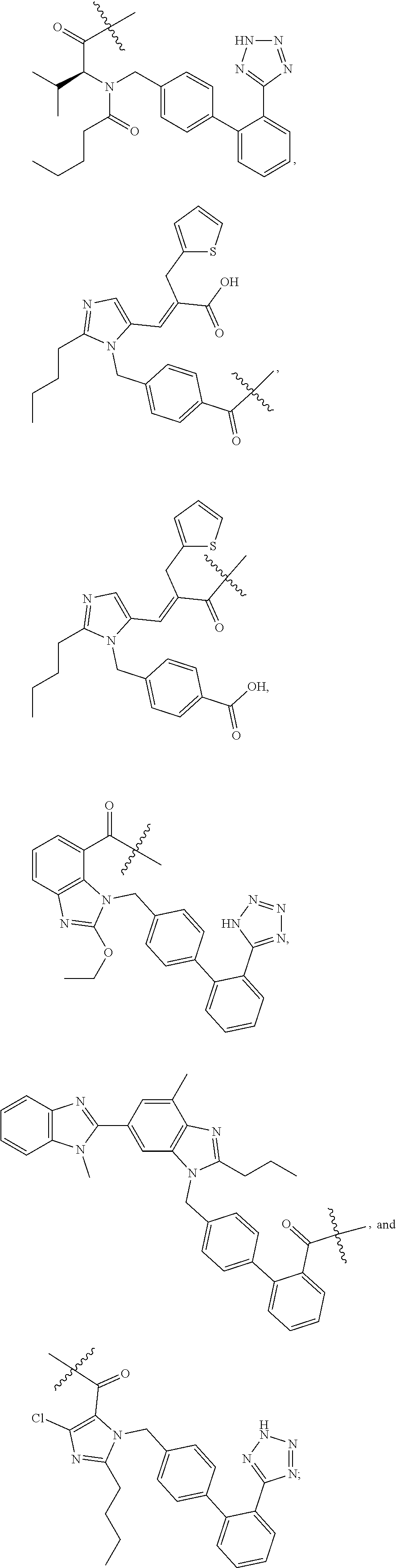

and

(b) mixing the compound of formula (XVa) or formula (XVb) with a compound of formula (X):

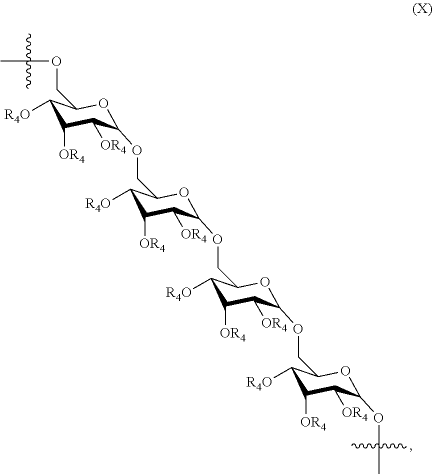

wherein R₄ is H, allyl carbamate, or mixtures thereof.

Another embodiment of the invention is a method for preparing the substituted dextrans of formula (I-1), which are functionalized with an ARB, comprising:

(a) transforming a compound of formula (XIVa) or formula (XIVb) into a compound of formula (XIVa-1) or formula (XIVb-1):

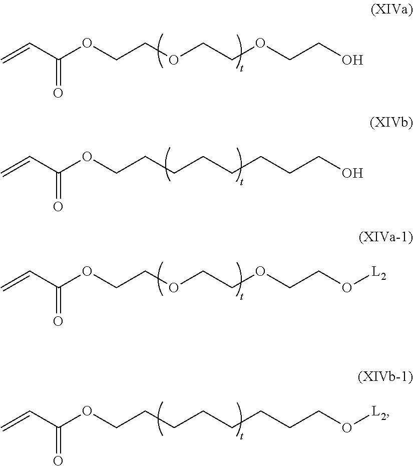

wherein t is 0 or an integer between 1 and 500, between 1 and 250, between 1 and 200, between 1 and 100, between 1 and 50, or between 1 and 10; and L₂ is a group selected from the group consisting of C₁-C₁₀ alkyl or

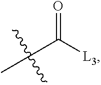

and wherein L₃ is C₁-C₁₀ alkyl;

(b) reacting the compound of formula (XIVa-1) or formula (XIVb-1) with a compound of formula (IIc):

Q₂-CH₂OH (IIc)

to yield a compound of formula (XIVa-2) or formula (XIVb-2):

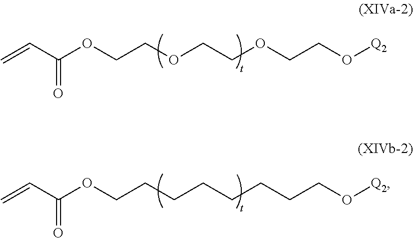

wherein Q₂ is

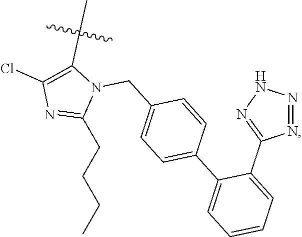

and wherein t and L₂ are as defined in step (a); and

(c) mixing the compound of formula (XIVa-2) or formula (XIVb-2) with a compound of formula (X):

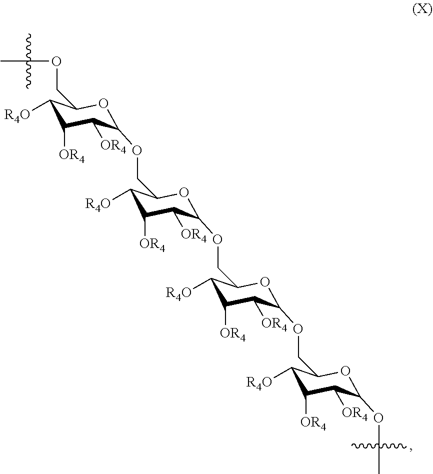

wherein R₄ is H, allyl carbamate, or mixtures thereof.

In an embodiment of this invention, the CIP-EMRs of the invention further comprise a fluorescent label selected from at least one dye with emission wavelengths in the range of 350 to 2500 nm.

In an embodiment of this invention, the CIP-EMRs of the invention are a high-viscosity solution comprising at least one substituted dextran that is functionalized with a fluorescent label, and/or at least one acrylate-comprising compound that is functionalized with a fluorescent label, and/or at least one acrylate-comprising compound.

In an embodiment of this invention, CIP-EMRs of the invention further comprising a fluorescent label facilitate measurement of wound healing progress via, for example, fluorescence microscopy studies measuring the change in fluorescence in the CIP-EMR and/or the surrounding wound environment.

This invention provides substituted dextrans of formula (I-2), which are functionalized with a fluorescent label, that may be used in the CIP-EMRs of the invention:

W-J-Y₁ (I-2),

wherein W is a fluorescent label selected from at least one dye with emission wavelengths in the range of 350 to 2500 nm;

J is

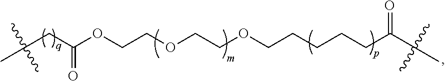

wherein q is 0 or an integer between 1 and 10, m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10; and

Y₁ is a dextran functionalized with ethylamine, allyl carbamate, or mixtures thereof.

This invention provides a method for preparing the substituted dextrans of formula (I-2), which are functionalized with a fluorescent label, comprising:

(a) reacting a fluorescent label of formula (XI-a) with a structure of formula (XIIa) to yield a compound of formula (XIIIa):

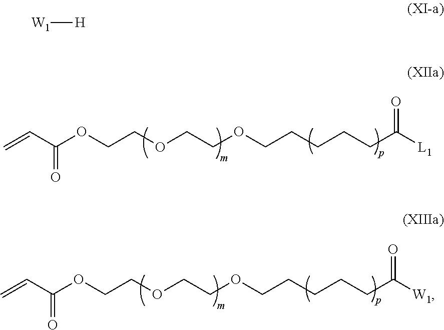

wherein m is 0 or an integer between 1 and 500, between 1 and 250, between 1 and 200, between 1 and 100, between 1 and 50, or between 1 and 10; p is 0 or an integer between 1 and 500, between 1 and 250, between 1 and 200, between 1 and 100, between 1 and 50, or between 1 and 10; L₁ is selected from the group consisting of —Cl, —Br, —I, and —ORₐ, wherein Rₐ is H, C₁-C₁₀ alkyl,

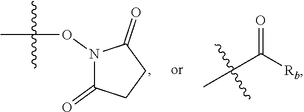

and wherein R_{b} is C₁-C₁₀ alkyl; m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10; and W₁ is selected from the group consisting of:

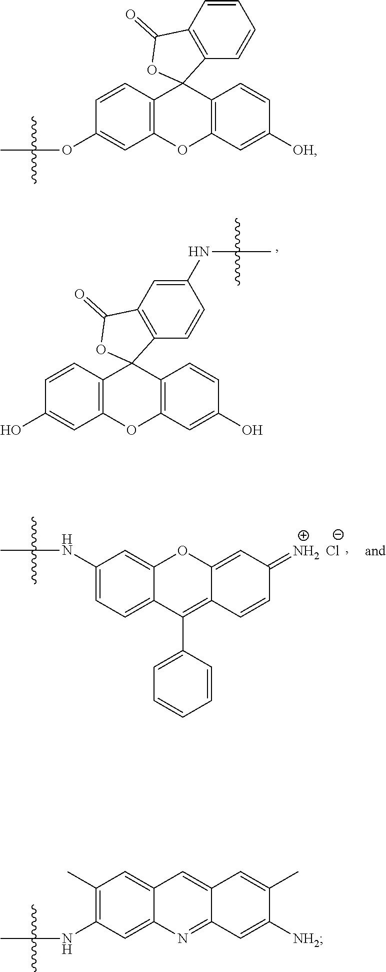

and

(b) mixing the compound of formula (XIIIa) with a compound of formula (X):

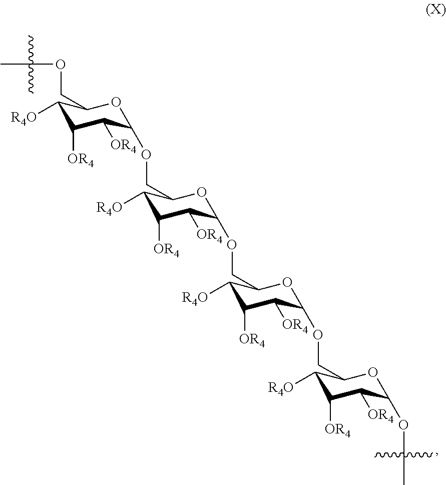

wherein R₄ is H, allyl carbamate, or mixtures thereof.

In another embodiment, the substituted dextran of formula (I-2), which is functionalized with a fluorescent label, is prepared by a method comprising the steps of:

(a) mixing a compound of formula (X):

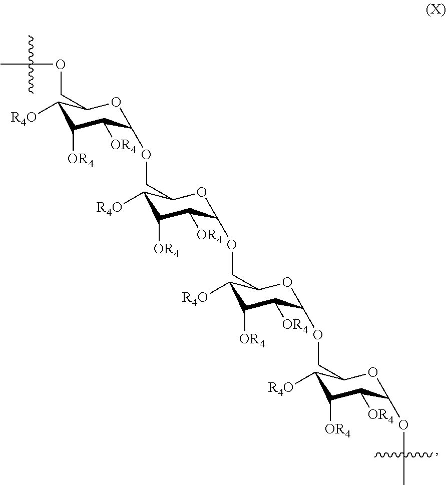

wherein R₄ is H, allyl carbamate, or mixtures thereof,

with a compound selected from the group consisting of polyethylene(glycol)diacrylate, acrylate-polyethylene(glycol)-succinimidyl valeric acid, polyethylene(glycol)acrylate, or mixtures thereof; and

(b) reacting the product of step (a) with a fluorescent label of formula (XI-a):

W₁—H (XI-a),

wherein W₁ is selected from the group consisting of:

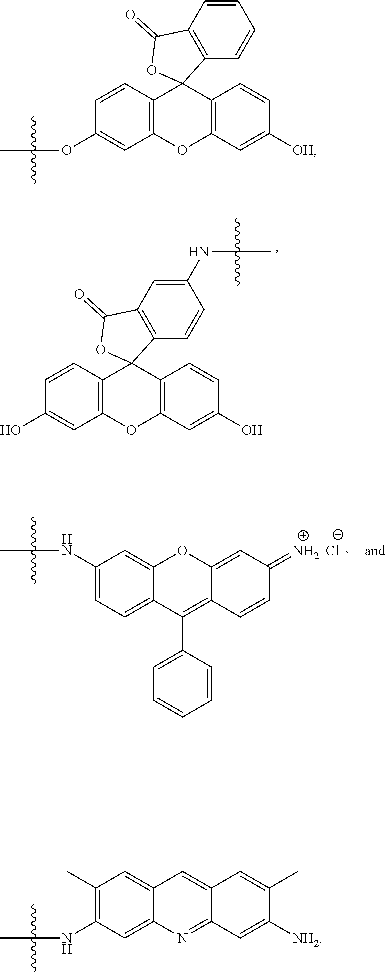

The CIP-EMRs of the invention also ensure patient compliance. The CIP-EMRs of the invention provide mechanical support to the wound and increase cell migration and revascularization of the wound site. The CIP-EMRs of the invention comprising ARBs further provide ARB release into the wound for a prolonged time period. The prolonged time period is at least 5 days, preferably at least 7 days, preferably at least 8 days, preferably at least 10 days, preferably at least 12 days, preferably at least 14 days, preferably at least 20 days. Thus, with the CIP-EMRs of the invention, patients also do not have to clean and apply a dressing or drug to a wound daily, which leads to increased patient compliance and faster healing.

This invention provides a method of treating wounds, comprising applying an effective amount of the CIP-EMRs of the invention to a wound in a patient in need thereof and curing the CIP-EMRs in the wound by exposing the CIP-EMR to UV-light and/or visible light for a suitable exposure time and intensity.

An effective amount of the CIP-EMR of the invention is an amount that fills a wound bed, and/or conforms to wound boundaries, and/or creates a uniformly-cured EMR upon exposure to UV-light and/or visible light. Further, an effective amount of the CIP-EMR is an amount such that wound healing occurs faster for wounds treated with the CIP-EMR than occurs for a control, e.g., an untreated wound or a pre-cured EMR without a drug. A patient in need thereof includes e.g., a mammal having a wound as described herein. The mammal may be, e.g., a primate, e.g., a human or a monkey, a horse, a cow, a pig, a dog, a cat, or a mouse.

Suitable UV-light and/or visible light exposure times for curing the CIP-EMR in the wound are those that create a uniformly-cured EMR and that do not inhibit the proliferation, migration, and survival of cell types that are critical to wound healing and/or that limit cytotoxicity.

Suitable UV light intensities for curing the CIP-EMR in the wound are those that create a uniformly-cured EMR and that do not inhibit the proliferation, migration, and survival of cell types that are critical to wound healing and/or that limit cytotoxicity.

In an embodiment of this invention, exposing the CIP-EMR of the invention to UV light is conducted using a commercially-available, handheld, long-wavelength UV lamp of adjustable intensities. Examples of suitable UV lamps are 365 nm handheld units. Other examples of suitable UV lamps are 400 W/200 WPI mercury lamps, which are capable of curing unfunctionalized EMRs in less than two minutes at a height of 6.5 inches above the material (dose=7 J/cm²), and 15 W benchtop units, which are capable of curing unfunctionalized EMRs in approximately thirty minutes at a height of 6 inches above the material (dose=10 MW/cm²). Sun et al., JBMA 2009. UV dosage is directly proportional to UV intensity (W/cm2) and time.

Suitable visible light intensities for curing the CIP-EMR in the wound are those that create a uniformly-cured EMR and that do not inhibit the proliferation, migration, and survival of cell types that are critical to wound healing and/or that limit cytotoxicity.

In an embodiment of this invention, exposing the CIP-EMR of the invention to visible light is conducted using a commercially-available, handheld, visible light lamp of adjustable intensities. Visible light dosage is directly proportional to visible light intensity (W/cm2) and time. Examples of suitable visible light lamps are lamps which have a wavelength of 415 nm and/or lamps which have an output power between about 20 mW/cm² and about 55 mW/cm² and/or lamps which apply a total dose between about 10 J/cm² and about 25 J/cm².

Another embodiment of the invention is a method of treating wounds with an effective amount of the CIP-EMRs this invention, wherein wound healing occurs within between 1 day and 100 days after applying the CIP-EMRs to the wound.

Another embodiment of the invention is a method of treating wounds with an effective amount of the CIP-EMRs of this invention, wherein wound healing occurs within between 1 day and 10 days after applying the CIP-EMRs to the wound.

Another embodiment of the invention is a method of treating wounds with an effective amount of the CIP-EMR of this invention, wherein the CIP-EMR is applied twice daily, once daily, twice weekly, once weekly, twice monthly, or once monthly.

Another embodiment of the invention is a method of treating wounds with an effective amount of the CIP-EMR of this invention, wherein the wounds are acute wounds or chronic wounds.

Another embodiment of the invention is a method of treating wounds with an effective amount of the CIP-EMR of this invention, wherein the wounds are excision wounds or burn wounds.

Another embodiment of the invention is a method of treating wounds with an effective amount of the CIP-EMR of this invention, wherein the wounds are diabetic ulcers or pressure wounds.

In another embodiment of this invention, the CIP-EMRs of the invention are applied to surgical wounds in sharp debridement procedures. For example, as a last step in a surgery, a patient receiving operating room wound debridement may have his or her wound filled with the CIP-EMRs of the invention. These CIP-EMRs in the wound are then cured with UV light and/or visible light. The treated wound is then dressed and monitored in the hospital clinic for healing.

Another embodiment of the invention is a method of treating wounds with an effective amount of the CIP-EMR of this invention, wherein the polymers of the cured EMR resulting from curing CIP-EMR with UV light and/or visible light are degraded by endogenous enzyme activity in the wound bed as healing proceeds.

This invention also provides a method of measuring the progress of wound treatment, comprising applying an effective amount of a CIP-EMR comprising a fluorescent label to a wound, curing the CIP-EMR comprising a fluorescent label with UV light and/or visible light, and measuring the amount of fluorescence in the cured EMR or in the surrounding wound via fluorescence microscopy wherein a change in the fluorescence in the wound is indicative of healing of the wound. Measuring may occur twice a day, once a day, twice a week, once a week, twice a month, or once a month.

In the methods of this invention, CIP-EMRs of this invention may be applied to the wound in the form of a pharmaceutical composition comprising the CIP-EMR and a pharmaceutically acceptable excipient.

The physical characteristics and wound healing effects of the CIP-EMRs of this invention may be assessed using any method discussed above for the interstitial EMR-drug compositions, EMR-drug conjugates, and/or EMR-fluorescent label conjugates.

The viscosity of partially-cured low-viscosity solutions of this invention is monitored over time after UV and/or visible light exposure to determine whether they continue to react after UV and/or visible light exposure. These partially-cured solutions are exposed again to UV and/or visible light to convert them from partially-cured solutions to fully-cured EMRs. The fully-cured EMRs are analyzed by testing the EMR's swelling ratio, measuring tensile strength, imaging with SEM, and calculating pore size and uniformity and comparing the results of these analyses to control samples (e.g., unfunctionalized, cured EMRs).

The cure rates of the CIP-EMRs of the invention are assayed in wounds of various sizes and shapes to define the relationship between wound size and curing parameters (e.g., UV and/or visible light strength and UV and/or visible light exposure time) that mimics controls (e.g., unfunctionalized EMRs). The cure rates are tested in vitro using polydimethylsiloxane (PDMS) molds of various sizes (up to 9 cm²), depths (up to 3 mm), and shapes (randomly generated). These molds are chosen to model clinically common full-thickness wound sizes.

The effect of mixing time and mixing temperatures on cure rates and outcomes of the CIP-EMRs of the invention is evaluated by varying the curing time (e.g., between 1 and 90 minutes) and/or by varying the curing temperature (e.g., between 4 C-37 C). The characteristics of the cured products resulting from the CIP-EMRs of the invention and their effects on wound healing are evaluated by, e.g., swelling ratio tests, tensile strength measurements, SEM imaging, pore-size and uniformity calculations, oxygen permeability, in wound degradation, cytotoxicity, fibroblast recruitment as described herein. Suitable unfunctionalized and/or functionalized, benchtop-cured (i.e., previously cured and not cured in place) EMRs are used as controls.

The CIP-EMRs of the invention are mechanically characterized by two methods: gelation viscosity and compressive modulus. The gelation viscosity is determined using a rheometer (e.g., a MCR 302 rheometer from Anton-Paar, Ashland, Va.) to measure changing viscosity and mechanics over UV and/or visible light exposure time. The compressive modulus, which is the destructive mechanical testing metric for quality control for unfunctionalized EMR products, is determined by unconfined parallel plate compression of the samples in a phosphate buffered saline bath using a dynamic mechanical analyzer (DMA) (e.g., a Q800 DMA from TA Instruments, New Castle, Del.). Suitable unfunctionalized and/or functionalized, benchtop-cured EMRs are used as controls.

After curing, the CIP-EMRs of the invention are tested for unreacted monomer content, aqueous swelling, DMA compression, and oscillating rheology. Suitable unfunctionalized and/or functionalized, benchtop-cured EMRs are used as controls. After curing, the products resulting from the CIP-EMRs of the invention have a Young's modulus between about 10 Pa and about 2000 Pa, between about 10 Pa and about 1500 Pa, between about 10 Pa and about 1000 Pa, between about 10 Pa and about 500 Pa, and between about 10 Pa and about 250 Pa. Further, after curing, the products resulting from the CIP-EMRs of the invention have full oxygen permeability under normoxic conditions.

### Definitions

As used herein, the following terms have the following meanings. If not defined, a term will have its accepted meaning in the scientific and medical community.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio.

“Substituted phenyl” refers to a phenyl group which is substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio.

A “pharmaceutically acceptable excipients” is any safe, non-toxic substance that may serve as a vehicle or carrier for the EMR-drug conjugates of the invention. A pharmaceutically acceptable excipient may also be any safe, non-toxic substance that is known in the pharmaceutical industry to be useful for preparing pharmaceutical compositions, including fillers, diluents, agglutinants, binders, lubricating agents, glidants, stabilizer, colorants, wetting agents, and disintegrants. The pharmaceutically acceptable excipient may be, for example, water, saline solution, and/or polyethylene glycol.

The term “degree of substitution” is explained as follows: Each monomer unit of unfunctionalized dextran has three hydroxyl groups. If the sum of the integrated intensities of the hydroxyl peaks is 11, and the integrated intensity of the anomeric proton is 4, the ratio of the integrated intensities of the hydroxyl to the integrated intensity of the anomeric proton is 2.75. This value (2.75) is subtracted from the total number of hydroxyls (3), to calculate the degree of substitution (3-2.75=0.25). The calculated value (0.25) for the degree of substitution also corresponds to an average of one substituted hydroxyl group for every 4 monomer units.

The term “unfunctionalized EMR” represents EMRs that do not comprise a drug and/or a fluorescent label. Suitable examples of unfunctionalized EMRs, as well as methods for preparing and characterizing unfunctionalized EMRs may be found in, e.g., U.S. Pat. Nos. 8,900,868 and 9,655,844, U.S. Pre-Grant Publication Nos. 2013/0102531 and 2015/0174154, and Sun et al., PNAS 2011, 108, 20976-20981 and Shen et al., Acellular Hydrogels for Regenerative Burn Wound Healing: Translation from a Porcine Model, Journal of Investigative Dermatology 2015.

The term “functionalized dextran” is a dextran that has at least one substituted hydroxyl group, wherein the substituent may be selected from a polymerizable compound.

The term “substituted dextran” is a dextran that has at least one substituted hydroxyl group, wherein the substituent may be selected from a small molecule comprising a carboxylic acid, from a fluorescent label, and mixtures thereof.

The terms “transformed” and “transforming” mean that a compound has been reacted with a suitable reagent to effect a desired chemical change. For example, a compound with an alcohol functional group may be transformed (i.e., oxidized) into a compound with a carboxylic acid functional group by reacting the compound with an alcohol functional group with a suitable oxidizing reagent. As another example, a compound with a carboxylic acid functional group may be transformed into a compound with an acyl chloride functional group by reacting the compound with a carboxylic acid functional group with a suitable chlorinating reagent (e.g., thionyl chloride). By seeing the structures of the starting compound (i.e., the compound to be transformed) and the transformed compound, a person of ordinary skill in the art would recognize the transformation and would recognize the reagents necessary to effect the transformation.

Polyethylene(glycol)diacrylate may be abbreviated as “PEGDA.” Polyethylene(glycol)diacrylate and PEGDA are used interchangeably herein.

Acrylate-polyethylene(glycol)-succinimidyl valeric acid may be abbreviated as “acrylate-PEG-SVA.” Acrylate-polyethylene(glycol)-succinimidyl valeric acid and acrylate-PEG-SVA are used interchangeably herein.

The foregoing description has been set forth merely to illustrate the invention and is not meant to be limiting. Since modifications of the described embodiments incorporating the spirit and the substance of the invention may occur to persons skilled in the art, the invention should be construed broadly to include all variations within the scope of the claims and equivalents thereof.

### EXAMPLES

### Example 1—EMR Functionalization with a Fluorescent Label

Dextramate was prepared by reacting dextran (70 kDa) with allyl isocyanate molecules in DMSO to produce a dextran that is functionalized with allyl carbamate groups. Subsequently, an aqueous solution of, dextramate was mixed with polyethylene(glycol)diacrylate and acrylate-polyethylene(glycol)-succinimidyl valeric acid in a polyethylene(glycol)diacrylate:acrylate-polyethylene(glycol)-succinimidyl valeric acid ratio of 80:18:2. The mixture was cured with UV light using standard curing conditions (e.g., curing at room temperature using a 15-W ballast from a height of 6 inches to deliver 15 mW/cm² over 30 minutes).

The cured product was reacted with 5-fluorescein amine. Without wishing to be bound by theory, it is contemplated that the N-hydroxysuccinimide groups of the cured product were replaced by the primary amine of 5-fluorescein amine.

### Example 2—Functionalization with an ARB

Polyethlene(glycol)acrylate (MW 5000) (0.002 mmol) was reacted with valsartan (0.02 mmol) in a ratio of 1:10 (moles polyethylene(glycol)acrylate: moles valsartan) in the presence of dicyclohexylcarbodiimide (DCC) (0.024 mol), dimethylaminopyridine (DMAP) (0.024 mmol), and dimethylformamide (DMF) (5 mL). The reaction was allowed to proceed overnight. The valsartan-functionalized polyethlene(glycol)acrylate was purified by dialysis in diH₂0 for 3 days at 3500MWCO. The purified valsartan-functionalized polyethlene(glycol)acrylate was reacted with EMRdextran PEGDA precursor mixture using cross-linking chemistry. Without wishing to be bound by theory, it is contemplated that the mechanism was photo-initiated radical cross-linking, that the initiator degraded and was covalently bound to the initiation site, and that the reaction proceeded via radical carbon-carbon bond formation until all sites were quenched.

### Example 3—Biocompatibility of the UV Curing of CIP-EMRs

To verify the biocompatibility of the UV curing of the CIP-EMRs of the invention, in vitro cell viability and proliferation are quantified under varying intensities of UV light.

A confluent layer of primary dermal fibroblasts (HDFn, ATCC) under standard cell culture conditions is exposed to increasing strengths of UV light over a range of time points. One set of primary dermal fibroblasts are exposed to UV light in the presence of the CIP-EMRs of the invention to determine if UV absorbance during curing protects the underlying cells from UV-induced cell damage. As a control, one set of primary dermal fibroblasts are exposed to UV light in the absence of the CIP-EMRs of the invention.

In vitro cell viability is quantified using a live/dead fluorescent assay (Calcein & Ethd-1, Invitrogen). The live/dead fluorescent assay detects cell lysis or death events caused by exposure to UV light.

UV exposure intensity is titrated down to identify the tolerance threshold of the cells (exposure conditions under which there is negligible adverse response). In vitro cell proliferation is quantified using a WST-1 proliferation assay (Abnova) against a control culture with no precursor or UV exposure.

### Example 4—Biocompatibility of the Visible Light Curing of CIP-EMRs

To verify the biocompatibility of the visible light curing of the CIP-EMRs of the invention, in vitro cell viability and proliferation are quantified under varying intensities of visible light.

A confluent layer of primary dermal fibroblasts (HDFn, ATCC) under standard cell culture conditions is exposed to increasing strengths of visible light over a range of time points. One set of primary dermal fibroblasts are exposed to visible light in the presence of the CIP-EMRs of the invention to determine if visible light absorbance during curing protects the underlying cells from visible light-induced cell damage. As a control, one set of primary dermal fibroblasts are exposed to visible light in the absence of the CIP-EMRs of the invention.

In vitro cell viability is quantified using a live/dead fluorescent assay (Calcein & Ethd-1, Invitrogen). The live/dead fluorescent assay detects cell lysis or death events caused by exposure to visible light.

Visible light exposure intensity is titrated down to identify the tolerance threshold of the cells (exposure conditions under which there is negligible adverse response). In vitro cell proliferation is quantified using a WST-1 proliferation assay (Abnova) against a control culture with no precursor or visible light exposure.

### Example 5—In Vitro Studies of Wound Healing with CIP-EMRs

Wound healing using the CIP-EMRs of the invention is assessed in a porcine excisional wound healing assay. Suitable unfunctionalized and/or functionalized, benchtop-cured EMRs are used as controls. In porcine excisional wound healing assays, macroscopic closure rate is quantified and the associated microscopic cellular mechanisms are determined.

Full thickness, 2.5 cm wounds are generated by circular scalpel excision after the dorsal-lateral area is shaved and sterilized. Alternatively, non-circular, irregularly shaped wounds are generated. Eight wounds (four per side) are generated between the crest of the shoulders and the coccygeal tuberosity. One side of the body (four wounds) is treated, and the other side of the body (four wounds) is the control. On one set of animals, unfunctionalized EMR is applied. On another set of animals, CIP-EMRs of the invention are applied.

To address the overall effect of the CIP-EMRs of the invention on healing, full-thickness excisional histology sections are taken at days 7, 14, 21, and at wound closure. All wounds are photographed and measured every three days during wound dressing changes.

Wounds treated with the CIP-EMRs show increased granulation tissue deposition, increased collagen organization, and complete re-epithelialization for 2.5 cm diameter, full-thickness excisional wounds as compared to an EMR control. The enhanced effects achieved by the CIP-EMRs are contemplated to be due to improved contact between the CIP-EMRs and the wound bed.

The microscopic and mechanistic performance of the CIP-EMRs of the invention is also evaluated. Histology sections, stained with Masson's Trichrome and H&E, reflect collagen deposition and remodeling, re-epithelialization, and closure rate.

Immunohistochemistry is used to stain for specific cellular markers associated with inflammatory responses and vascular reconstruction in the wound bed. Stains are also performed for macrophages (M1/M2, EMR1), neutrophils (MPO), keratinocytes, and vascular markers (CD31). qRT-PCR is also conducted on tissue removed from the center of the wound and wound edge to quantify specific markers in the wound healing arrays. Tensiometry testing (Electromatic, Cedarhurst, N.Y.) is also performed on the healed skin at closure to determine the elasticity and strength of the healed skin.

### Example 6—In Vitro Studies of Wound Healing with CIP-EMRs

Cytotoxicity of the CIP-EMRs of the invention is evaluated by culturing human fibroblasts with the CIP-EMRs of the invention in wells and then quantifying fibroblast viability, morphology, and proliferation in the presence of the CIP-EMRs of the invention.

Keratinocytes and neutrophil-like cells are cultured individually and in co-culture. CIP-EMRs of the invention are cured in several small, sterile PDMS molds with sterile-filtered precursor solution using various handheld UV lamps. A controlled number of cells are seeded onto the cell scaffold (keratinocytes) and/or into the surrounding media. Cellular infiltration and migration is characterized using time-lapse contrast microscopy. The rate of CIP-EMR degradation is characterized by measuring the mass of CIP-EMR over multiple time-points in culture.

Quantitative real-time PCR (qRT-PCR, LifeTech, Carlsbad, Calif.) is used to quantify levels of predictive wound healing markers (e.g. MMPs, TGFβ, etc.) in the cell culture and surrounding media. The ratio of the mass of the dry starting material to the mass of the freeze-dried degraded material is calculated to determine the rate of scaffold degradation over time.

### Example 7—Preparation of an EMR-Drug Conjugate Comprising Valsartan Entrapped in EMR

Dextramate (24.2 mL of a 23.1 w/w % solution), PEGDA (16.3 mL of a 8.6 w/w % solution), Irgacure (14 mL of a 0.5 w/w % solution), and valsartan (2.82 mL or 1.41 mL of a 24.8 w/w % solution or 2.82 mL of a 1:10 dilution of a 24.8 w/w % solution) are mixed together. The mixture is cured for 10 minutes. After curing, 100 mL of dH₂O is added to the cured product to create a swelled gel. Unexpectedly, valsartan does not exhibit reactivity under the curing conditions.

1. A cure-in-place extracellular matrix replacement (CIP-EMR) comprising a high-viscosity solution comprising:
(a) at least one functionalized dextran, wherein the functionalized dextran is a dextran functionalized with ethylamine, allylcarbamate, or mixtures thereof; and
(b) optionally comprising an acrylate-comprising compound selected from the group consisting of polyethylene(glycol)diacrylate, polyethylene(glycol)acrylate, and acrylate-polyethylene(glycol)-succinimidyl valeric acid, and mixtures thereof.

2. The CIP-EMR of claim 1, further comprising one or more UV-crosslinking catalysts and/or one or more visible light-crosslinking catalysts.

3. The CIP-EMR of claim 1, further comprising a functionalized acrylate-comprising group, wherein the functionalized acrylate-comprising group is polyethylene(glycol)acrylate functionalized with an angiotensin receptor blocker (ARB).

4. The CIP-EMR of claim 3, wherein the ARB is selected from the group consisting of valsartan, olmesartan, azilsartan, eprosartan, candesartan, telmisartan, carboxylosartan, irbesartan, losartan, or mixtures thereof.

5. The CIP-EMR of claim 4, wherein the functionalized acrylate-comprising compound is polyethylene(glycol)acrylate functionalized with valsartan,

6. A method of preparing a CIP-EMR of claim 1, comprising preparing a low-viscosity solution comprising the functionalized dextran and converting the low-viscosity solution into a high-viscosity solution.

7. The method of preparing a CIP-EMR of claim 6, wherein the low-viscosity solution further comprises:
(a) at least one acrylate-comprising compound selected from the group consisting of polyethylene(glycol)diacrylate, polyethylene(glycol)acrylate, and acrylate-polyethylene(glycol)-succinimidyl valeric acid;
(b) a UV-crosslinking catalyst; and/or
(c) a visible light-crosslinking catalyst.

8. The method of preparing a CIP-EMR of claim 6, wherein the low-viscosity solution is converted into a high-viscosity solution by increasing the concentration of the functionalized dextran and/or the acrylate-comprising compound.

9. The method of preparing a CIP-EMR of claim 6, wherein the low-viscosity solution is converted into a high-viscosity solution by adding a high-viscosity, non-irritating polar solvent and/or solvent additive.

10. The method of preparing a CIP-EMR of claim 6, wherein the low-viscosity solution is converted into a high-viscosity solution by partially curing the low-viscosity solution with UV-light and/or visible light.

11. A method of treating wounds, comprising applying an effective amount of the CIP-EMR of claim 1 to a wound in a subject in need thereof and curing the CIP-EMRs with UV-light and/or visible light for a suitable exposure time and using a suitable UV light intensity and/or visible light intensity.

12. The method of treating wounds of claim 11, wherein the CIP-EMR is applied to the wound twice daily, once daily, twice weekly, once weekly, twice monthly, or once monthly.

13. The method of treating wounds of claim 11, wherein the wounds are acute wounds, chronic wounds, excision wounds, burn wounds, diabetic ulcers, or pressure wounds.

14. A method of treating wounds, comprising applying an effective amount of the CIP-EMR of claim 3 to a wound of a subject in need thereof and curing the CIP-EMRs with UV-light and/or visible light for a suitable exposure time and using a suitable UV light intensity and/or visible light intensity.

15. The method of treating wounds as in claim 14, wherein the CIP-EMR is applied twice daily, once daily, twice weekly, once weekly, twice monthly, or once monthly.

16. The method of treating wounds as in claim 14, wherein the wounds are acute wounds or chronic wounds, excision wounds, burn wounds, diabetic ulcers, or pressure wounds.

17. An extracellular matrix replacement (EMR)-drug conjugate of formula (I):
Q-X—Y (I),
wherein Q is a small molecule comprising a carboxylic acid group, X is a linker group, and Y is an EMR.

18. The EMR-drug conjugate of claim 17, wherein the linker group is selected from the group consisting of 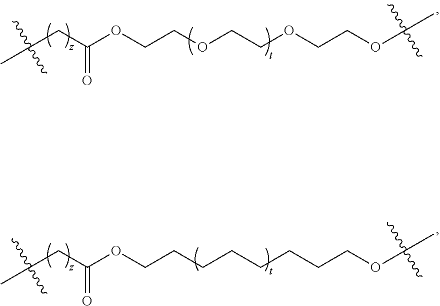
and mixtures thereof, wherein t is 0 or an integer between 1 and 500, between 1 and 250, between 1 and 200, between 1 and 100, between 1 and 50, or between 1 and 10; and z is 0 or an integer between 1 and 10.

19. The EMR-drug conjugate of claim 17, wherein the EMR is prepared by curing an unfunctionalized dextran, functionalized dextran, and mixtures thereof.

20. The EMR-drug conjugate of claim 19, wherein the functionalized dextran is a dextran functionalized with ethylamine, allyl carbamate, or mixtures thereof.

21. The EMR-drug conjugate of claim 20, wherein the small molecule comprising a carboxylic acid group is an angiotensin receptor blocker (ARB).

22. The EMR-drug conjugate of claim 21, wherein the ARB is valsartan.

23. A method of making an EMR-drug conjugate comprising:
(a) reacting a small molecule of formula (XVIII) with a compound of formula (XIXa) or formula (XIXb) to yield a compound of formula (XXa) or (XXb): 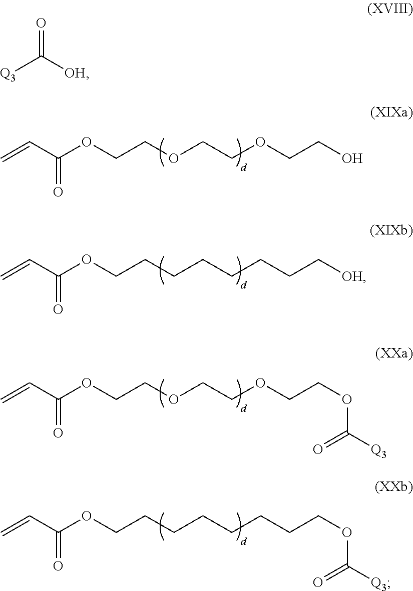
wherein Q₃ is a residue of a small molecule, and
wherein d is 0 or an integer between 1 and 500, between 1 and 250, between 1 and 200, between 1 and 100, between 1 and 50, or between 1 and 10;
(b) reacting the compound of formula XXa or formula XXb with a dextran of formula X: 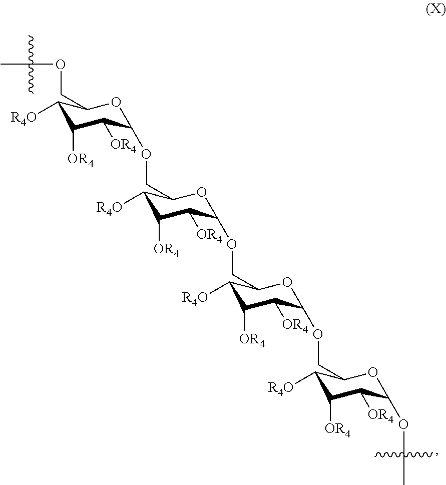
wherein R₄ is H, allyl carbamate, or mixtures thereof;
(c) optionally adding to the product of step (b) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and
(d) curing the product of step (b) or step (c) with UV light and/or visible light.

24. The method of claim 23, wherein Q₃ is selected from the group consisting of 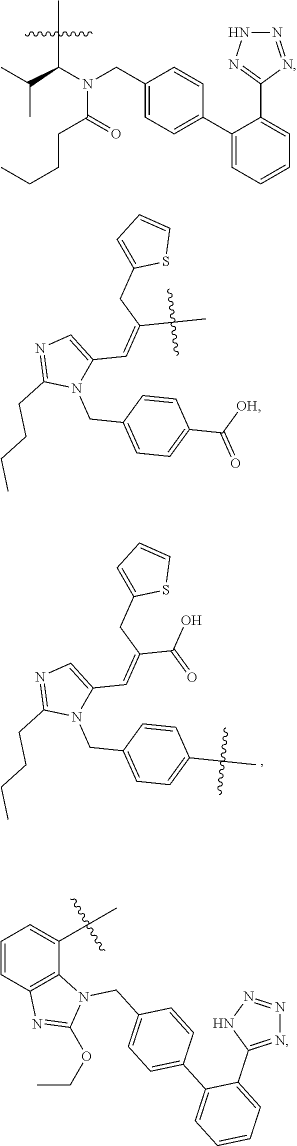 [SMILES: *[C@H](C(C)C)N(Cc1ccc(-c2ccccc2-c2nn[nH]n2)cc1)C(=O)CCCC ; */C(=C/c1cnc(CCCC)n1Cc1ccc(C(=O)O)cc1)Cc1cccs1 ; *c1ccc(Cn2c(/C=C(\Cc3cccs3)C(=O)O)cnc2CCCC)cc1 ; *c1cccc2nc(OCC)n(Cc3ccc(-c4ccccc4-c4nnn[nH]4)cc3)c12] 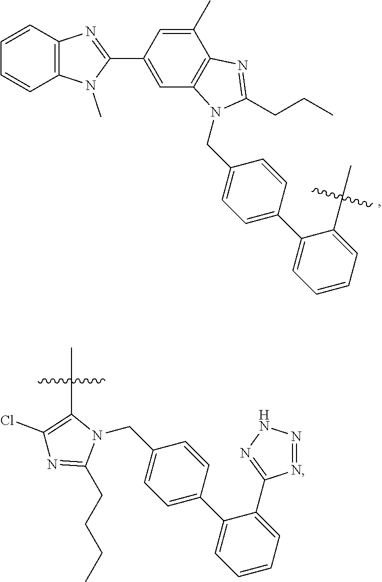 [SMILES: *c1ccccc1-c1ccc(Cn2c(CCC)nc3c(C)cc(-c4nc5ccccc5n4C)cc32)cc1 ; *c1c(Cl)nc(CCCC)n1Cc1ccc(-c2ccccc2-c2nn[nH]n2)cc1]
and mixtures thereof.

25. A method of treating wounds in a subject in need thereof comprising applying an effective amount of an EMR-drug conjugate of claim 17 to a wound.

26. The method of treating wounds of claim 25, wherein the EMR-drug conjugate, is applied twice daily, once daily, twice weekly, once weekly, twice monthly, or once monthly.

27. The method of treating wounds of claim 25, wherein the wounds are acute wounds or chronic wounds, excision wounds, burn wounds, diabetic ulcers, or pressure wounds.

28. An EMR-fluorescent label conjugate of formula (XI):
W-J-Y (XI),
wherein W is a fluorescent label, J is a linker group, and Y is an EMR.

29. The EMR-fluorescent label conjugate of claim 28, wherein the fluorescent label is a dye with emission wavelengths in the range of 350 to 2500 nm.

30. The EMR-fluorescent label conjugate of claim 28, wherein the linker group is 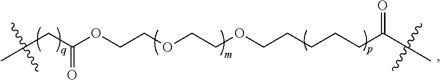
wherein q is 0 or an integer between 1 and 10, m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10.

31. The EMR-fluorescent label conjugate of claim 28, wherein the EMR is prepared by curing a unfunctionalized dextran, functionalized dextran, and mixtures thereof.

32. The EMR-fluorescent label conjugate of claim 31, wherein the functionalized dextran is a dextran functionalized with ethylamine, allyl carbamate, or mixtures thereof.

33. The EMR-fluorescent label conjugate of claim 28, wherein the fluorescent label is 5-fluoresceinamine.

34. A method for preparing the EMR-fluorescent label conjugate of claim 28, comprising:
(a) reacting a fluorescent label of formula (XI-a):
W₁—H (XI-a)
with a structure of formula (XIIa): 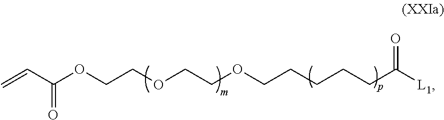
wherein L₁ is selected from the group consisting of —Cl, —Br, —I, and —ORₐ, wherein Rₐ is H, C₁-C₁₀ alkyl, 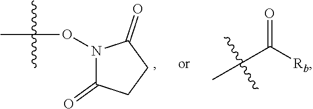
and wherein R_{b} is C₁-C₁₀ alkyl; m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10,
to yield a compound of formula (XIIIa): 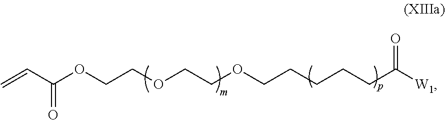
wherein W₁ is selected from the group consisting of: 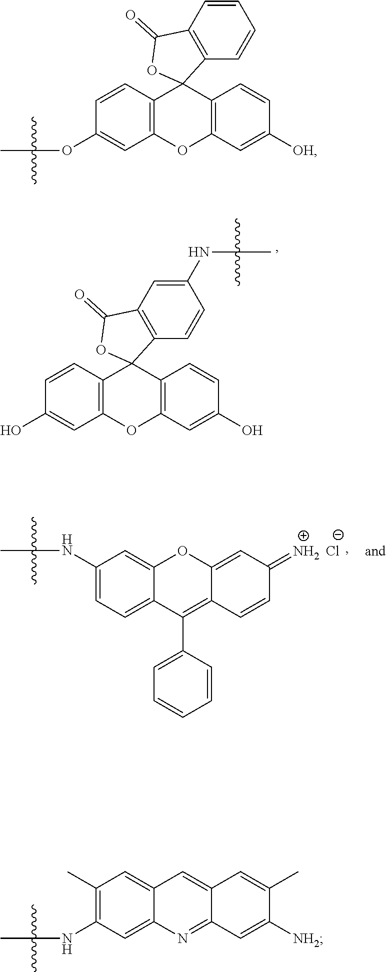
m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10;
(b) mixing the compound of formula (XIIIa) with a compound of formula (X): 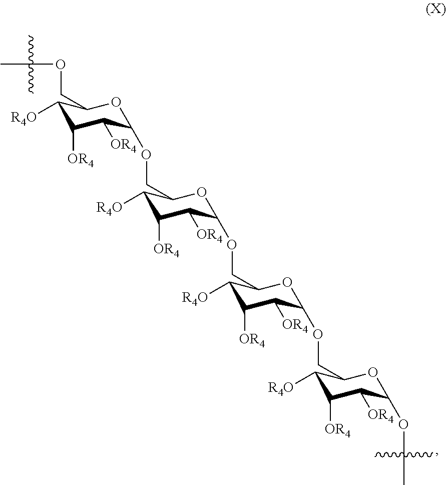
wherein R₄ is H, allyl carbamate, or mixtures thereof;
(c) optionally adding to the product of step (b) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and
(d) curing the product of step (b) or step (c) with UV light and/or visible light.

35. A method for preparing the EMR-fluorescent label conjugate of claim 28, comprising:
(a) mixing a compound of formula (X): 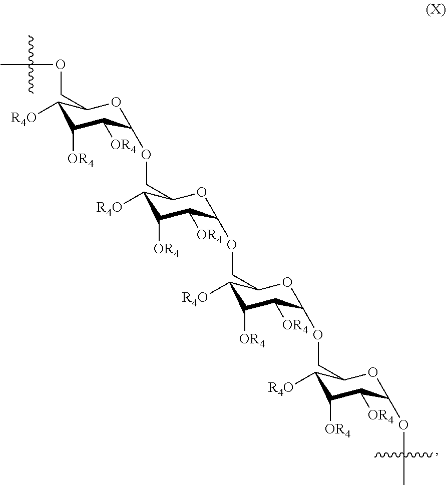
wherein R₄ is H, allyl carbamate, or mixtures thereof,
with a polymerizable compound selected from the group consisting of polyethylene(glycol)diacrylate, acrylate-polyethylene(glycol)-succinimidyl valeric acid, polyethylene(glycol)acrylate, and mixtures thereof;
(c) curing the product of step (a) with UV light and/or visible light; and
(d) reacting the product of step (c) with a fluorescent label of formula (XI-a):
W₁—H (XI-a),
wherein W₁ is selected from the group consisting of: [chemical structure]
